SEQUENCE LISTING (30 sequences)

SEQ ID NO: 1 - DNA, 624 bp, Cricetulus griseus
  source 1..624: mol_type = genomic DNA
atggctgtgctccgttccttgctgccacagctggggctgtttctgtgcctggctctgtgcttttctcctgccttgtctgcaagttataatgacccctgcatggtctttgatacaatctccaccagcgacaacttgggagtcagcattacggtggccgggagctctggtgagaacataacctacacagtgttggttcacgtgaacagttccgtcagtgccgtgattctgaaagcagtgaaccagaacaagcccgtgggttcctgggttggagcaactcaggaatgcaatgacagcagtgtcctatatcgcgtgacaccctcagacagttctggcttccaggcaacgtggatagttcctaattctgaggatatgactaaagtcaacctgcatgtcttcttagctaccggcaatggaacagctgcaatgacatctgtgaacctgggagagcaaaaaacacctgtaaccgttaaccccacccctgagatttctgagaccaaccaaaccacagccatgactacagacaggaccacagccaaaagcctggctgtcaatgctctcggcagccccctcgcaggtgccttccacatcctgcttgtttttctcatcagcaaactcctcttctag

SEQ ID NO: 2 - DNA, 228 bp, Cricetulus griseus
  source 1..228: mol_type = genomic DNA
atgataaatactagcagggcctcactcctggtcttaaaactttgccacattctgtcttacataatacagaatcaacccagacacgacctgatcagtcagtccagttcaaagaaacttacatctgtaagagtgaagaaatgtccattccgtgagaatttgcaaggcaccattagttggtggtcaatgaaatttttaaaaacacctggtctcatcaagtgttcaatataa

SEQ ID NO: 3 - DNA, 162 bp, Cricetulus griseus
  source 1..162: mol_type = genomic DNA
atgtggtcagagaagacagagcatcctccttacacacagagcgtagtgctttggggaaaaaagtcaactttacttaccagagaaaccattaccattgatgaaataaagtcatttcatcgtttactgcacaagattcatttgaagggtatgtatggatattaa

SEQ ID NO: 4 - DNA, 1872 bp, Cricetulus griseus
  source 1..1872: mol_type = genomic DNA
atgagaggtcttttctggccactgtcactgttgctgctctccctccagccctgggaaatccagctccagtctgcaggtcctaggtgctatactgggcccctggatttggtgttcgtgattgatagctcccgcagcgtgcgccccttcgagttcgagaccatgcggcagtttctagtgggcctcctccgtagcctggatgtggggctgaacgccacgcgtgttggagtgatccagtattctagccaagtgcagagcgtgtttcccctgggcgccttctcccgtcgcgaggacatggagcgcgccatcagcgccgtagtgcctctggcgcagggcaccatgaccgggctggcgatccagtatgccatgaacgtggccttcagtgaggccgaaggcgcccgcccaccggaggagcgagtgccgcgggtcttggtcatcgtgaccgacgggagacctcaagaccgagtggccgaagtggccgcgcaggcgcgcgcccgcggcatcgagatctacgcggtgggggtgcagcgagccgatgtgggctctctacgagccatggcttcgccgccgctggatcagcacgtcttcttggtggagtccttcgatctcatccaggagtttggtctgcagtttcagggtcggctgtgcgggaaggacctgtgtgctgagtggggacatggctgccaacaccagtgtgtcaatgctccaggaaccttctactgcacctgcaactctggctacaagctagccccagataataagaactgtttggccatggacctctgtgctgaaggaacccacggttgtgaacacctctgtgtcaattccctggactcctatttctgtcgttgtcgagctggctttgcactccagcaggaccagaagagctgcagggccattgactactgtagctttggaaaccatagctgccagcatgagtgtgtgaacaccctagaggggccccagtgtatctgcagagagggtcatgacctgctgcctgatgggaggagctgtcgggtcagggacttctgcaatggtgtggatcatggctgcgagttccagtgtgtgagtgagggtctttccttccgctgcctgtgccctgaggggaggcgacttcaggctgatggcaagagctgtgaccggtgccgagagggccacgtggatcttgttcttctggtggatggctccaagagtgtgcgtccacagaacttcgagctggtgaaacgcttcgtgaaccagatcgtggacttcctggacgtgtcccctgagggcacacgcatcgggctggtgcagttctccagccgggtgcgcaccgagttcccactgggccgctatggcactgcagctgaggtgaagcaggcagtcctggctgtggagtacatggagcgcggcaccatgacgggactggcgctgcgccacatggtggagcacagcttctcagaggcgcagggtgcgcggcctcgtgacctcaacgtgcctcgtgtgggcttggtgttcactgatggccgctctcaggatgacatttcagtgtgggcagctcgtgccaaagaggaaggcatcgtcatgtatgctgtgggcgtgggcaaggccgtggaggaagagctgcgtgagatcgcatccgaaccttcggagctgcatgtgtcctacgctccggacttcagcaccatgactcatctgctggagaacctcaaaggcagcatttgcccagaggagggcatcagcgctgggacagagcttcggagtccctgcgaatgcgaaagcctcgtggaattccagggccgcacgctgggggcgctcgagagcctgacacggaacctggcccagctgaccgagcgcctggaggaactggagaaccagctgactagccggaagtga

SEQ ID NO: 5 - DNA, 153 bp, Cricetulus griseus
  source 1..153: mol_type = genomic DNA
atgctgttctcaagtgcacctgtaactcctaatgttttctttacctgcgaatggagtgaacaaggtaggacaaaccccagacccgcaggacatgctgggacacctgggaggccaaggtctgcagttggaaacctgagtatctctgagacttga

SEQ ID NO: 6 - DNA, 264 bp, Cricetulus griseus
  source 1..264: mol_type = genomic DNA
atgacgttctccaccaagaaggtccgactctgccggttccgccgaacatgctccttagctagcggccaacagaaagaaaaaggaacaagcgtaagaaaacttggagaggaaaaagcgaccattgtctgcagctgctggagacaacagcacggtgggtgcagggacggaatgtttgcggacacttggccaataggattaataaacacgacctcaaaaaccagaaagccagtcagtccctcgtccttggcccaggcccagctgtaa

SEQ ID NO: 7 - DNA, 2712 bp, Cricetulus griseus
  source 1..2712: mol_type = genomic DNA
atgagggatctgccaacactgccttgccactgggtttgtctactctggtccttctgcagctttcaggtgtgtggcacccagcccagagcccaggaacatccagggtttgcagtcttggcttctgcttctcattactggccattggaaaatgtggacgggatccttgagcttcaggacacaactggagcgttgcgaacccacaacctcactgtgcctccctcccacaatgctacctttgtctttaccaatgattctgcctactccaacttctctgcgactgtagatattatggaagggaaagtcaacaaaggtatttacctcaaggaggagaagggtgtgacattcctctactatggcagctacaagtcatcctgtatcagcaacccggcccagtgtggacctgagggagtgacattttcattcttctggaagactcaagaggagcagcctcgaccagccccctacgcttatgggggacaggtcatctctgatggtttcaaagtctgctctagtggtggaaagggctctgtggagctgtacacccgggataattccatgacatggaaggccaccttcaaccctcctggtccttactggactcatgtcctgttcacgtggaaatccaaggagggcctaaaagtctatgttaatgggactctgagcacctcagacccgagcgggaaagtgtctcacacctatggtgagccccatgtcaacctggtcatagggtctgagcaggaccagaccaagcgctacgagaacggagctttcgatgagttcatcatctgggaacgtgcactcactcccgatgagatcaagatgtacttcacagctgctattggtaaacactctctgttgtcttcaacaccaccagcgatgcctacagcacactccatggtgcccacggatgcctaccacccaatcatcaccaacctgacagaggagaggaaatgcttccagagacctgaaaccgtgcttcgataccttcaaaatgtgtccctcagcttgcccaataaatccctctcagaggaaacagcgctaaacctcacacagaccttcttaagaactgtgggtgaggtgcttctactgcccagctggacccatgtatcagaggacaacaccatgacgctgggcctggttgacaccatcgataccgtcatgggtcacatatcagccaacctgcagtccagagaaccccatatcaccctcacaggctcttcctccatggcagatttcttcgtggccaaggtccttcccccagcactaagtgccccccactatagattcccagcccatggtcacagctacattgagattccaagagaggcccttcacggcaaagcctggaccacgattgtcggccttctctaccactccatgcactattacctgaacaacatccccccagccagcacaaagattcctgaggctgtgaactgcagagactgcctgctgtctgtcgccagccacctgatatccctggaggtgtccccaccgcccactctgtcccagaacctatcgggctctcctctgatcactgtccatctcaggcacaaactgactcataagcagtatgtcgatgccaccaacgagagcaacgacctcttcctgtattgtgccttccttaacttcagctccggggaaggcatctggtcaagccagggctgtgcgctcagagagggaaacctcacccactcagtgtgccactgcacacatctcaccaactttgccatcctcatgcaagtggtcccactacagctcacccgtggacaccaggtggcgctgtcgtccatcagttatgttggctgctctctgtcagttctctgcctggctgccacgctggtcaccttcgcagtactgtcatctgtcagcaccattcggaaccagcgttaccacatccatgccaatctatccttcgctgtcctggtggcccaggttctgctgctcatcagcttccgtgtggagcctggcacagtcccgtgccaggtgctggccatcctcctgcactacttcttcctgagtgcctttgcttggatgctggtggagggactacatctctacagcatggtgataaaggtcttcgggtcagaggacagcaagcatctctactactatgggataggctgggggtgccctctcctcatctgcatcatctccatatcatcctctatggacagttatgggacaaatgacagttgctggctgtcactggggagtggtgccatttgggcctttgtgggccctgccctgctggttattgtggtcaacattgttattctggtagctgtgacaagggtcatttctcatgtcagcaccgacaactacaagatacatggggaccccagtgccttcaagttgacagccaaggctgttgctgtgttgctgcccatcctgggaacctcatgggtgtttggggtacttgccgtcagtgaccgggccctggtcttccaatacatgtttgctgtactcaactccttacagggcctcttcatcttcctctttcactgtctcttaaactcagaggtgagagcggccttcaagcataagaccaaggtctggtccctcacgagcagctcagcccgtaccacaaacaccaaacccttcagttccgatatcgtaaatggcacccggccaggcacagcatctacaaagttaagcccatgggacaagagcagccactctgcccaccgtgtggacctatcggctgtatga

SEQ ID NO: 8 - DNA, 6060 bp, Cricetulus griseus
  source 1..6060: mol_type = genomic DNA
atgggggccatgacctggttattgccaggcatctttctagctttgcttgccctcaattccgaaggtgcagtcctcaagaaagtcatccggcacaagcgacagagtggactgaacatgacccttccagaggagaatcagccagtggtgttcaaccatgtctacaacatcaagctgcctgtgggctctcagtgctcagtggatctggagtcagtgaatggggagaaagacctgaccccgacgccagagtcaagtggaaccttccaggaacatacagtggatggggaaaaccagattgtgttcacacaccgcatcaacatcccccgtagggcctgtggctgtgctgcagctcctgatgtgaaggagctcctgagcaggctggaggagctggagatgctggtgtcttctctacgggagcagtgcaccatgggtacaggctgttgtctccaacctgcagaaggccgtctggacactaggcccttctgcagtggccgaggcaacttcagtgccgaaggatgtggctgtgtttgtgagccaggctggaaaggccccaactgctctgagcctgaatgtcctggaaactgcaacctacagggccagtgccttgatggacagtgtgtctgtgacgagggtttcactggtgaggactgcagtcagctagcctgtcccaatgactgcaatgaccagggcagatgtgtgaatggagtctgtgtgtgctttgaaggctatgcaggggttgactgtggcctggaagtctgccccgtgccctgcagtgaggagcacgggacctgtttggatggcaggtgtgtgtgcaaagatggctttgctggtgatgattgcaatgagcccctgtgcctcaacaactgctacaaccgtgggcgatgtgtagagaacgagtgtgtgtgtgatgagggcttcacaggcgaggactgcagtgagctcatctgccccaatgactgcttcgaccgcggtcgctgtgtcaatggcacctgctactgtgaggaaggttttacaggtgaagactgtggtgagctcacctgccccaataactgccagggccatggccagtgtgaggagggacagtgtgtttgtgatgagggctttgccggtgcagactgcagtgaaaagcggtgtcctgaagattgtcaccaccgtggccgctgcctcaatgggcaatgtgagtgtgatgatgggttcatgggggctgactgtggggatctacagtgtcccaacggctgcagtgggcatggccgctgtgtcaatggacaatgtgtgtgtgatgagggctacactggagaagactgcagtcagcagcgatgtcccaatgactgccacaaccgaggcctgtgcgtacagggcaagtgcatatgtgaacaaggcttcaagggctttgactgtagtgagatgagctgtcccaatgactgccatggccatggccgctgtgtaaatggcatgtgcatctgtgatgatgaatacactggggaagactgcagagaccatcgctgtccccgggactgcagccagcggggacgctgtctggatggacaatgtatatgtgaggatggctttactggccctgactgtgctgagctctcctgccccaatgactgtcatggccatggccgctgtgtgaatggccagtgcatctgccatgagggcttcactggcaaagactgcaaagagcaaaggtgccccagtgactgccatggccaaggccgctgtgaggatggccagtgcatctgtcatgaaggcttcacaggcctggactgtgggcagcgctcctgccccaatgactgcagcaaccaagggcaatgtgtttcaggccgctgcatctgcaatgaaggttacagaggggaagactgttctgaagtgtctcctcccaaagaccttattgtaacagaagtaacagaagagaccgtaaacctggcctgggacaatgagatgcgggtcaccgagtacctcattatgtacacacccacccatgctgatggcctagagatgcagttccgtgtgcctggggaccagacatccaccactattcgagagctagagccaggggtggaatacttcatccgtgtgttcgccatcttggagaacaagaggagcatccctgtcagtgccagagttgctacctacttgcctgcacctgaaggactaaaattcaagtctatcaaggagacatctgtggaagtagagtgggaccctctagacattgctttcgaaacctgggagatcatcttccggaatatgaataaagaagatgagggagaaatcaccaaaagcttgaggagaccagagacctcttaccgacaaactggccttgctcctgggcaagaatatgaaatatctctgcacattgtgaaaaacaacaccagaggccctggcttgaagaaagtgaccacaacccgcttggatgcccccagccagattgaggtgagagatgtcacagacaccacagcactgatcacctggttcaagcccctggctgagattgatggcatcgagctctcctatggcatcaaggacgtgcctggggaccgcaccaccatcgatctcacacatgaagagaaccagtactccattgggaacctgaagcctgacactgagtatgaggtgtcccttgtctcccgaagggtggacatggcaagcaaccctgccaaggagaccttcaccacaggcctggatgcgcccaggaatcttcgccgtgtctcccagacagataacagcatcaccctggaatggaggaatgtcaaagctgacattgacagttacagaattaagtatgcacctatctcgggaggtgaccatgctgaggttgatgttccaaagagccaacaagctacaaccaaaaccacactcacaggtctgaggccaggaactgaatatggcgttggtgtttctgctgtgaagggagacaaggagagtgacccagcaaccatcaatgctgccacagaaattgatgcacccagggactttcaggtatctgaaaccacacaggacagtctgactctactctggaaaacaccactggccaagtttgatcgataccgcctcaactacagcctccccacaggccagtcaatagaggtccagttgccaaaggatgccacctcccatgtcctgacagatctggagccagggaaagaatatactgtcctcctcactgccgagaagggcaggcacaagagcaagcctgcacgtgtgaaggcatccacggaacaagctccttccctggaaaatctcaccgtgactgaggcaggctgggatggcctcagactcaactggactgcggatgacttggcctatgagtactttgtcattcaggtgcaggaagccaacaaggtggaggctgctcacaacttcacagtgctcggcaacctccgggctacagacatcccgggtctcaaggctgccactccctatagagtctccatctatggggtagcccggggctctagaacaccagtgctctctgctgaggcttccacagggaaaactcccaatttgggagaggtctctgtcgctgaggtgggctgggatgccctcaaactcaactggactgctccagaaggggtctatcagaactttttcattcaggtgctagaggctgacacgacccagaccgtccagaacctcacagtccctggaggactgaggtcagtggacctgcctgggctcagagcagccactcactatcacatcaccatccgaggggtcactcaggacttcagcacagcccctctctctgttgaagtcttaacagaggagctccctcatctgggaggcttatcagtgactgaggtcagctgggatggcctcatactcaactggaccacagacgatctggcctatgagcactttgttatccaggtgcaggaggccaacaatgtggaggctgctcagaacctcacagtgcccggtagcatcagggctatggaaatcccaggcctcaaggctgccactccttatagagtctccatctatggggtgatccagggctatagaacaccaatgctctctgctgacgcctccacagccaaagaacctgaaattggaaacttgaatgtttctgacgtaactcctgagagcttcaatctctcctggacagcgactgatgggatcttcgacatgtttactattgaaattattgattccaataggttgctgcagatggcagagcataacatatctggtgctgaacgaactgcccacatctcagggcttccccctagtactgatttcattgtctacctctctggaattgctcccagcatccggaccaaaaccatcagtaccacagctaccacagaagctgaaccagaagttgacaaccttctagtttcagatgccactccaggtggtttccgtctgtcctggactgctgatgaagggatattcgacagttttgttctcaggattagagataccaaaaagcagtccgaaccacaagaaataatcctcccttcccctgaacgtaccagggacataactggtctcagagaggccactgagtatgaaattgaactctatggaataagcggtggaaggcgatcccagccagtcagtgccatagcaacaacagccatgggttctccgaaggaaatcatgttctcagacatcactgacaatgcagccacagtcagctggaaggcacccactgcccaagtagagagcttccggattacctatgtgcctatgacaggaggtgccccctccatggtgacagtggatggaactgatactgagacccgtctggtgaagctcacccctggggtggaataccatgtcagcgttattgccatgaagggctttgaagaaagtgatcctgtctcggggtcactaaccacagctctggatggtccgtctggcttgctggcagcaaacatcacagacacagaagccttggcactgtggcaaccagccattgccactgtggatagttatgtcatctcctacacaggggagagagtgccagaagttacacgcacagtttctgggaatacggtggagtatgagctacatgacctggagcctgccacagaatacacactgaggatctttgcagagaaaggacaccagaagagctctgctattactaccaagttcaccacagaccttgattccccaagagacttgactgctactgaggttcagtcggaaactgccctcctcacctggcgacctccccgggcatcaatcaccggttacctcctggtctatgaatctctggatggtacagtcaaggaagtcattgtgggaccggataccacctcctacagcctggcagacctgagtccatccacccactacacagtgaggatccaagcattgagtgggtccctaaggagcaagttgatccaaaccatctttaccacaattggactcctgtacccattccccagggattgttctcaagcaatgttgaatggagacaccacctctggcctctacaccatctatataaatggtgacaagactcaagctctagaagtctactgtgatatgacctctgatggaggtggatggatcgttttcctgagacgcaaaaatggacgtgaggacttctatagaaactggaaggcctatgctgctggttttggggaccgcagagaagaattctggcttggactggataacctgagcaaaatcacagcccaagggcagtatgagctccgggtggacctacaagaccatggggaatcagcctttgctgtgtatgataggttcagtgtgggagatgccaagagtcgctacaagctgaaagtagaaggatacagtggaacagcaggtgactccatgaactaccacaatggcagatccttctccacctatgacaaggacacagactcagccatcaccaactgtgctctgtcctacaaaggagctttctggtataagaactgccatcgtgtcaacctaatgggcagatatggggacaataaccacagtcagggcgttaactggttccattggaagggccatgagtactcaatccagtttgctgagatgaaactgagacccagcaacttccgaaatcttgaaggcaggcggaagcgagcataa

SEQ ID NO: 9 - DNA, 312 bp, Cricetulus griseus
  source 1..312: mol_type = genomic DNA
atgaccaacagccagatgaggtggtcatatccaaatcaggctttagctgaccagctaccaaacaaaagttcgcagcatcagtttgcttcctttcatgggagaacagaattagaagccaagatcccggtgctacacctgtttattgctgctggagtgacattcctttcagatcctccaagcttcaatgcaaagcatcacatcagcccaggcgtaagccctcatctgagaaatcccaccaccgtctgcacccatgttaaggggacgggtcctagtgtctccagctctaacccattgccaagtggaccgtgctaa

SEQ ID NO: 10 - DNA, 4401 bp, Cricetulus griseus
  source 1..4401: mol_type = genomic DNA
atgatgagctttgtgcaaagtgggacttggcttcttctcgccctgcttcatcctactttcatttgggcacagcagtccagtgtagacgaatcgggatgcagccaccttggtcagtcctatgagtccagggatgtttggaagccagaaccatgtcaaatatgtgtctgtgactccggatctgtcctctgcgatgacataatatgtgatgaggatccactagactgccccaacccagagatcccatttggagaatgttgtgcaatatgcccacagccttcaacagctgctcctgtacctcctgatggtcacggacctcaaggccccaaaggagatccagggcctcctggcattcctgggagaaatggtgatcctggccttccagggcaaccaggtctccctggacctcctggctctcctggaatctgtgaatcatgtccaactggtggtcagaattattctccccagtatgactcatacgatgtcaagtctggagtaggagcaggaggacttggtggtggctatcctggtccagctggtcccccaggccctcctggtccccctggctcagttggacatcctggctcccctggttctcctggataccaaggtccccctggtgaacctggtcaagctggtccttcaggccccccaggacctcctggtgctattggtccatctggtcctgctggaaaggatggagagtcaggaagaccgggacgacctggagaacgtggactgcctggtcctccaggtatcaaaggcccaagtggcatgcctggattccctggtatgaaaggacacagaggttttgatggacgaaacggagaaaagggtgaaaccggtgctcctggattgaagggtgaaaacggtctcccaggtgacaatggagctcctggccccatgggtcccagaggggctcctggtgagagaggacggccaggccttcctggagctgcaggtgctcgaggcaatgatggtgctcggggcagtgatggccaaccaggtccccctggtcctcctggaaccgcaggattccctggatcccctggtgctaagggtgaagttgggcctgcagggtcccctggctcaaatggctctccaggacaaagaggggaacctggaccacagggacacgctggtgctcaggggcctcctggccctcccgggaataatggcagtcctggtggcaaaggtgaaatgggtcctgctggcattcctggagctcctggactaatgggagctaggggtccccctggaccagctggcactaatggtgcacccgggcaacgaggtccttcaggtgaacccggcaagaatggtgccaaaggagagccaggagctcgtggtgaacggggggaagctggttccccaggaatcccagggcctaagggtgaagatggcaaagatggatcacctggagaacctggtgcaaatggacttccaggaactgctggagaaaggggtgctcctggcttccgaggacctgcagggccaaatggcatcccaggagaaaagggtcctgctggggagcgtggtgccccaggtcctgcagggccccgaggagtggctggagaacctggccgagatggaaacccaggaggcccaggaatgaggggtgtgcccggaagcccaggaggaccaggcaatgatgggaaaccaggacctcccggaagtcaaggagaaagtgggcgccctggtcctcctggtccatctggcccccggggtcagcctggtgtcatgggtttccctggccctaaaggaaatgatggtgctcctggcaagaatggagaacggggtggccctggaggacctggccttccaggtcctgctggaaagaatggtgaaactggacctcagggtcccccaggtcctactggcccatctggtgacaagggagaatctggaccccctggtccacaaggattacaaggaatacctggtaccagtggtcctccaggagaaaatggaaaaccaggtgaaccagggccaaagggagaagttggtgcacctggagttcccgggggcaagggtgatgccggtgcccctggagaacgtggaccacctggaactgcaggggtccctggtcttagaggaggagctggaccccctggccctgagggaggaaagggccctgctggcccccctggtcctcctggtacttctggttctcctggtctacaagggatgcctggagagagaggaggtcctgggagtcctggtccaaagggtgaaaagggtgaaccagggggtgccggtgctgacggagctccaggaaaggatgggccaaggggtcctactggtcctattggtccccctggcccagctggtcagcctggagataagggtgaaggtggtgcccctggacttccgggtatagctggacctcgaggtggccctggtgagagaggtgaacatgggcctccaggacctgctggcttcccgggtgctcctggacagaatggtgaaccaggcgctaaaggagaaagaggtgcccctggagagaaaggagaaggaggccctcctggacctgcaggacttcctggaggttctggacctgctggtcctcctggtcctcaaggtgtcaagggtgaacgtggcagtcctggtggtcctggtgctgctggctttcctggtggacgtggtcttcctggtcctcccggcaacaatggtaacccaggccccccagggcctagtggtgctcctggcaaggatggtcctccaggtcctgctggtaacagtggttcccctggcaacccgggagtagctggaccaaaaggtgatgctggtcagcctggagagaagggaccacctggtgctcaaggccctccgggatctccaggcccacttggaattgcaggacttacaggagcacgaggtcttgctggaccaccaggcatgccaggtcctaggggtagccctgggcctcaaggtatcaagggtgaaagtggaaaaccgggagccagtggccataatggagaacgtggtcctcctggaccccaaggtcttcctggtcaacctggtacagctggtgaacccggaagggatggaaaccctggatcagatggtcagccaggacgagatggatctcctggtggcaagggtgatcgtggtgaaaatggctctcctggtgccccaggcgctcctggtcatccaggaccacctggtcctgttggtccagctgggaaaagtggtgacagaggagaaacggggcctgctggtccttctggtgctccaggtcctgctggatctcgcggtcctcccggtccccaaggtcctcgaggtgacaaaggtgaaactggtgaacgtggctccaatggcatcaaaggacatcgaggattccctggcaatccaggtcccccaggttctcctggtgctgctggtcaccagggtgcagttggtagtcctggacctgcaggtcccagaggaccagttggaccacatgggcctcctggaaaagacggaactagtgggcatccaggtcccattggaccaccaggacctcgaggcaacagaggtgaaagaggatctgaaggctcaccaggccaccctgggcagccaggcccccctggacctcctggtgcacctggtccttgctgtggtggaggtgctgctgcccttggaggtggtggtgaaaagtctggtggattttcaccatattatggagatgaaccaatggatttcaagattaacaccgaggagattatgtcttcactcaagtctgttaatggacaaatagaaagcctcattagtcctgatggttctcgtaaaaaccctgctcgcaactgcagagacctgaaattctgccatcctgatctcaagagcggagaatattgggttgatcctaaccaaggttgcaagatggatgctatcaaagtattctgtaacatggaaaccggggagacatgcataaatgctagtcctatgactgtgccacggaagaactggtggacagatgcctctgctgaaaagaaacatgtttggtttggagaatctatgaatggtggatttcagttcagttatggcaatcctgatcttcctgaagatgtccttgatgtacagctggcttacctcagactcctctccagccgggcttcccagaacattacataccactgcaagaacagcattgcctacatggatcaggccagtggcaatgtaaagaagtctctaaagctgatgggatcaaatgaaggggaattcaaggctgaaggaaacagcaaattcacttacacagttctggaggatggctgtgctaaacacactggggagtggagcaaaacagtcttcgaataccgaacgcgcaaggccctgagactgcccatcatagatatcgcaccctatgacatcggtggtcccgatcaagaatttggtgtggacattggccctgtttgctttttataa

SEQ ID NO: 11 - DNA, 666 bp, Cricetulus griseus
  source 1..666: mol_type = genomic DNA
atggcgggaaagccagtccttcactactttgatggcgggggcagaatggagcctgtccggtggctcctggctgcagcgggagtagagtttgaagaaaaatttctgaaaactcgggatgacttggcaaggttaagaaatgatgggagtttgatgttccagcaagtgcccatggtggagattgacgggatgaagctggtgcagaccagagccattctcaactacattgcctccaaatacaacctctatgggaaggacatgaaggagagagccctcattgacatgtatgcggaaggtatagcagatctggatgaaatagttctccatcaaccttatattccccaagaggagaaagaggcaaaccttgccaagatcaaggacaaagcaaggaaccgttacttccctgcctatgagaaggtgttaaagggccatggacaagattatctcgttggcaacaggctgagcagggctgatgtttacctggttgaacttctctaccatgtggaagagctggaccccagcgttttggccaacttccctctgctgaaggcactgagaaccagagtcagcaacctccccacagtgaagaagtttcttcagcctggcagccagaggaagccttatgaggatgagaaatgtgtagaatcagcaatgaagattttcagttag

SEQ ID NO: 12 - DNA, 1824 bp, Cricetulus griseus
  source 1..1824: mol_type = genomic DNA
atgtggttacggtctctggtcaaacagttagagcgaggggaagcctctgtggtcgatcttaagaagaacctggaatatgcagcaacagtgctagaatctgtgtacattgatgaaacaaggaggcttctggatacagaggatgagcttagtgacattcagtcagatgctgtgccttctgaggtccgagactggctggcctccaccttcacacgacagatggggatgatgctaaggagaagtgatgagaagcccaggttcaagagcatcgtccatgcagtgcaagctgggatatttgtggagagaatgtataggcggacatcaaacatggttgggttgagttatccaccagctgttattgatgcgttaaaggatgtggacaaatggtccttcgatgtcttttctctcaatgatgccagtggagatcatgcgctgaagttcattttctatgaattactcactcgctatgacctgatcagccgttttaagatccccatttctgcacttgtctcatttgtggaggccctggaagtggggtacagcaagcacaaaaacccttaccataacctgatgcatgccgctgacgtcacccagactgtgcattacctcctctataagacaggagtagcaaactggctgacagaactggagatcttcgcaataatcttctcggctgccatccatgactatgaacacaccggaactaccaacaatttccacattcagacccggtcggatccagctatcttgtacaatgacagatctgtgctggagaatcaccacttgagtgcagcttaccgccttctgcaggaagacgaggagatgaatattctggtcaacctctcaaaggatgactggagggagtttcgaaccttggtaattgagatggtaatggccacggatatgtcctgtcatttccagcaaatcaaagccatgaagacagccctgcagcagccagaagcaattgagaagccaaaagccttatctctgatgctgcacacagcagacatcagccatcctgcaaaagcatgggacctgcaccaccgctggaccatgtctctcctggaggagttcttcagacagggtgacagagaagcagagctggggctgccattttctcctctatgtgacagaaagtcaaccatggttgctcagtcacaagtgggttttattgacttcattgtggagcccaccttcactgtgctcacggatatgactgaaaaaattgtgagtccattaatcgatgaaacatcccagactggtgggacaggccagaggcgctcaagtttgaacaacatcagtgcctcagatgcaaagcgaccaggtgtcaagagttctgggtcagaaggaagcgctcccatcaacaattctgtcatccctgttgactataagagttttaaagccacctggactgaggtggtgcacgtcaatcgggagcggtggcgggccaaggtgcccaaagaagagaaagccaagaaggaagctgaagagaaggctcgcctggctgctgaggaaaagcaaaaggaaatggaagcccaagaccaaactgaacaaggcaaagctgagaaaaagacatctggagaaacgaaaggccaagtcaatggagcgcgtacaaacaaggggaagagccccaaaggtgacaaggccggagagaaacagcagaatggtgacttgaaagagggtaaaaataaggcagataagaaggatcactccagcaccggaaatgattcaaagaaaacagatggtacaaagaagcgttctcatgactcacctgctccaagcactagttccacaagtcgcattaccttgccaggagactatgggtaa

SEQ ID NO: 13 - DNA, 1599 bp, Cricetulus griseus
  source 1..1599: mol_type = genomic DNA
atggaccgcgcggggtgtctgggtgcgggcctgcggggactctgcgtggctgcgctcgtgctcgtgtgcgccgggcacggggcacgccgcgaggacgggggaccagcttgctatgggggattcgatctctacttcatcctggacaagtcaggaagcgtgctgcaccactggaatgaaatctactacttcgtggagcagctggctcatagattcatcagcccacagctgaggatgtccttcatcgtcttctctactcgagggacaaccttaatgaaactaactgaggacagggaacagatccgacaaggcctagaagaactccagaaagttctgccaggaggagacacttacatgcatgaaggatttgaaagggccagtgagcagatttactatgaaaacagccaaggatacaggacagcgagtgtcatcattgcactgacagatggggaactgcatgaagatctcttcttctactcagagagggaggctaacagatcccgaaaccttggcgcaattgtttactgtgttggtgtgaaggatttcaatgaaactcagttggctcggattgcagacagtaaggaccatgtgtttcctgtgaacgatggcttccaggctcttcaaggcatcatccactcaattctaaagaaatcctgcatcgaaattctagcggctgaaccatctaccatatgcgcaggagaatcttttcaagttgttgtaagaggaaacggcttccgacacgcccgcaacgtggacagggtcctctgcagcttcaagatcaatgactcagtcacactcaatgagaagccctttgctgtggaagacacttacttgctgtgcccagcacccatcttgaaagaagttggcatgaaagctgcactgcaggtcagcatgaatgatggcttgtctttcatctccagttccgtcatcatcaccaccacacactgttcagatggctccatcctggcaattgccctgctgatcctcttcctgttgctggccctggcactgctctggtggttctggcctctctgctgcacagtgatcatcaaggaggtccctccaccccctgttgaggagagtgaggaagaagacgatgatggcctgccaaagaagaaatggccaacagtagatgcctcttactacggaggacgaggagtcggaggcattaaaaggatggaggttcgctggggagagaagggctccacagaagaaggtgctaagttggaaaaggcaaagaatgcaagagtcaagatgccagagcaagaatatgagttcccggagccccgcaatctcaacaacaacatgcgccggccctcttccccccggaagtggtactcgcccatcaagggaaaactcgatgccttgtgggttctgctaaggaaaggctatgaccgagtgtccgtgatgaggccacagccgggagatacggggcgctgcatcaacttcaccagagtgaagaacactcaggcagccaagtacccactaagaaggttccagagatggctacactgccaagatgctctcaaccagattgtgtcccatggagaccaggaagaaagtcatttccgagaacggaatgcagcattggataagaaataa

SEQ ID NO: 14 - DNA, 678 bp, Cricetulus griseus
  source 1..678: mol_type = genomic DNA
atggcgtttacaagtatgtccacagagaagagtaagtgctctgggagggtccggcacaagctaattgtgatcggctcaccttccacagcactggctccggggttctactcaggccaagcagtctctgggctccagaaggtggtcttcgtcggcctgcttgggctcctcatagcgtctggtttttcttacactatcaacagcaacaatgaaagcaacgtaggaggaagtggtccgcaggcagtgagcatcaacaaccagcacaatgtggccaacgtcgacagcaacaatggctggggctcctggaatgccctctgggattatgaaaatagttttgctgcaaccagaatctttgccaagaaatcgtgcattgtgcacaaaatgaacaagaatgtcatgccctctcttcaagaactcgacacactggtcaaggagcaaaaggataaagagcctgaaggagcaactcccaaggaattgatgttctccatcaaccctaccagagtggaggacctgagtacattcgggccaaagattgctggcatgtgccagggcatcccaacctatgtagccgaggagattccaggaccgaaccagcctttgtacgcacagaagtgcttcactgctaatatactgtggatcatcaagttgtccttctgtggaacgtcagaggaaacatattag

SEQ ID NO: 15 - DNA, 924 bp, Cricetulus griseus
  source 1..924: mol_type = genomic DNA
atggccatggaccagagcacgcgggatgctaggcatccctcagttactgtgcgccctgcggatgctgcgctgcctgcggttgctgcgctcctcgcagaaacagtgcgtccagcggatgttgagagcactgaggatgctgcagaccccagggttaatgttcaagaccccgaggccgcgttgctgtccgccccgcactcgtgtttccgctgtcagctgctctactgggtaggcgccttgatgttgctgctcggggttgtctgtgttccgatcgccctcttcacccgcatcccgacccggccggctctcacagtcaccacctcgcctaccccggggaactcagagaagccttcaaacctgacggtcactcctgttcctcgaaatagctgcccgaaggctacagggcaggggtcttccgtgttcgccattcttcaggctaaagaggaagtaaagctggagcccaataggatcctgaagtggcatagccaagaaggagctgggatctcaaaaccgctccagggtctgaggtacgacaacgtcacaaacgagttggtggtgaacgaaagtgggctctactatgtgattttgcaactgaagctcaggcctgtgttaaaaaacacagaccgcaaggtgcggggtcaggtctctcttgttctgcaactgaatcccccgatagagcgccctgacaacttggccctgactgtggacctattcccttgctccatggagaccaacttagtggaaggctcctggagtcatctgatacccctgaaggctgacgaccgcctcagtgtgaatctgcgagcctatctgtatggagctcaggaggcatacaaagactgggagctctcccagactgctatcaccagctttgtgctcttccttgtgccaactgacaccccacaggaattgccatccatacgataa

SEQ ID NO: 16 - DNA, 3081 bp, Cricetulus griseus
  source 1..3081: mol_type = genomic DNA
atgactgctatcaagatgcttcagggttcttttcctgtgttcctgctgggggcgctcttgggagtcctccatgctcagcagcaggaagtcatctcacccgacatctctaccattgacaggaacaacaactgtccagagaaggctgactgcccagtcaatgtgtacttcgtgttagacacctcagagagcgtggccatgcagtcccccacagacagcctgctttatcatatgcaacagtttgtgcctcagttcatcagccagctgcagaacgaattttacctggaccaggtggccctgagctggcgttacggtggcctgcacttctccgaccaggtggaggtgttcagcccaccaggcagtgaccgggcctccttcactaagagcctccaaagcatccgctccttccgcagaggcacctttactgactgcgccctggccaacatgacacagcagatccggcagcatgtaggccgaggggtggtcaactttgctgtggtcatcaccgatggccacgtcacaggcaatccgtgtgggggcatcaaaatgcaagctgagcgcgcccgtgaggagggtatccggctctttgctgtggcccctaacaggaacctaaatgagcaaggcctgcgagacatcgctaacaccccacatgagctctaccgaaacaactatgccaccatgaggcctgactctactgagattgaccaggataccatcaaccgcattatcaaggtcatgaaacatgaagcctatggagagtgctacaaggtgagctgcctggagattcctgggccccatggacccaagggctaccgaggacagaagggtgccaagggcaacatgggtgagccaggagagcctggacagaagggtcgacagggagaccctggcatcgaaggccccattggattcccaggacccaagggtgtacctggcttcaagggagagaagggtgaatttggatccgatggtcggaagggagcccccggcctagctggcaagaatggaactgatggacagaagggcaaactgggccgcattgggcctcctggttgcaagggagacccaggaagtcggggccccgatggataccctggagaagccggaaccccgggcgagcaaggagaccaaggtgccaagggggactctggccgcccaggacgcaggggaccaccaggagatcctggtgacaagggaagcaagggatatcaaggcaacaacggagctcctggaagtccgggagtgaaaggaggcaagggagggcctggcccacgtggaccaaaaggagagccgggacgcaggggagatcccggaaccaagggcggcccaggaactgatggtccaaagggggagaagggagaccctggtcctgagggacctcgaggcctggctggagaagttggcagcaaaggagccaagggagacagaggtttgcctggacccagaggcccccagggggctcttggggagctgggaaaacagggatctaggggagaccctggtgatgctggaccccgtggagattcaggacagccaggacccaagggagatcctggaaggcctggattcagctacccaggaccccgaggaacacctggtgaaaaaggagagcccggtccaccaggccctgagggaggccgaggagactttggtctgaaaggagcacctggaaggaagggagacaagggagaaccagctgatcctggtccccctggtgaacctggccctcggggcccaagaggaatcccaggacctgagggagaacccggccctcctggagaccctggtctcacggaatgtgacgtcatgacctatgtgagggaaacctgtgggtgctgcgactgtgagaagcgttgtggtgctctggatgtggtcttcgtcatcgatagctctgagagtatcggctacaccaacttcaccttggagaagaactttgtcatcaatgtggtcaacaggctgggtgccattgccaaggaccccaagtctgagacaggcacacgtgtaggtgtggtgcagtacagccacgaaggcacttttgaagccatccggctagacgacgaacgagtcaactccctgtccagtttcaaggaggctgtcaaaaacctcgagtggattgctggcggcacctggacaccctctgccctcaagtttgcctacaatcagctcatcaaagagagccggcgccagaagactcgggtgtttgcagtggtcatcacagatgggcgccatgacccccgagatgacgacctcaatcttcgggcgctgtgtgatcgagatgtcactgtgacggccattggcattggtgacatgttccatgagacgcatgagagtgagaacctctactccattgcctgtgacaagccacagcaggtgcgcaatatgacacttttctctgacctggtggctgagaagttcatcgatgacatggaagatgtcctttgcccagacccccagatcgtgtgtccagaacttccctgccaaacagagctatatgtggcccagtgcacacagcggcctgtggacattgtcttcctgctggatggctcggaacggttgggtgagcagaacttccacaaggcacggcgcttcgtggaggaggtgtcacggcgcctgactctggcacgaaaggatgacgacccactcaacgcccgcatggccctgttgcagtacggcagccagaatcagcagcaggtggtcttcccactgacctacaacttgaccaccatccatgaggcactggagaggaccgcctacctcaattccttttctcacgtgggtgcaggcatcgtgcatgccatcaacaacgtggttcggggcgcacggggcggggcacggcgccacgcagagctctcctttgtcttcctcaccgatggcgtcacgggcaatgacagcctggaggagtcggtgcactccatgcgcaagcagaacgtggtgcccaccgtggtcgctgtgggcagcgatgtggacatggatgtgcttaccaagatcagcctgggtgacagggcggccatcttccgggagaaagactttgacagtctggcccagcccagcttcttcgacaggttcatccgttggatctgttag

SEQ ID NO: 17 - protein, 207 aa, Cricetulus griseus
MAVLRSLLPQLGLFLCLALCFSPALSASYNDPCMVFDTISTSDNLGVSITVAGSSGENITYTVLVHVNSSVSAVILKAVNQNKPVGSWVGATQECNDSSVLYRVTPSDSSGFQATWIVPNSEDMTKVNLHVFLATGNGTAAMTSVNLGEQKTPVTVNPTPEISETNQTTAMTTDRTTAKSLAVNALGSPLAGAFHILLVFLISKLLF

SEQ ID NO: 18 - protein, 623 aa, Cricetulus griseus
MRGLFWPLSLLLLSLQPWEIQLQSAGPRCYTGPLDLVFVIDSSRSVRPFEFETMRQFLVGLLRSLDVGLNATRVGVIQYSSQVQSVFPLGAFSRREDMERAISAVVPLAQGTMTGLAIQYAMNVAFSEAEGARPPEERVPRVLVIVTDGRPQDRVAEVAAQARARGIEIYAVGVQRADVGSLRAMASPPLDQHVFLVESFDLIQEFGLQFQGRLCGKDLCAEWGHGCQHQCVNAPGTFYCTCNSGYKLAPDNKNCLAMDLCAEGTHGCEHLCVNSLDSYFCRCRAGFALQQDQKSCRAIDYCSFGNHSCQHECVNTLEGPQCICREGHDLLPDGRSCRVRDFCNGVDHGCEFQCVSEGLSFRCLCPEGRRLQADGKSCDRCREGHVDLVLLVDGSKSVRPQNFELVKRFVNQIVDFLDVSPEGTRIGLVQFSSRVRTEFPLGRYGTAAEVKQAVLAVEYMERGTMTGLALRHMVEHSFSEAQGARPRDLNVPRVGLVFTDGRSQDDISVWAARAKEEGIVMYAVGVGKAVEEELREIASEPSELHVSYAPDFSTMTHLLENLKGSICPEEGISAGTELRSPCECESLVEFQGRTLGALESLTRNLAQLTERLEELENQLTSRK

SEQ ID NO: 19 - protein, 903 aa, Cricetulus griseus
MRDLPTLPCHWVCLLWSFCSFQVCGTQPRAQEHPGFAVLASASHYWPLENVDGILELQDTTGALRTHNLTVPPSHNATFVFTNDSAYSNFSATVDIMEGKVNKGIYLKEEKGVTFLYYGSYKSSCISNPAQCGPEGVTFSFFWKTQEEQPRPAPYAYGGQVISDGFKVCSSGGKGSVELYTRDNSMTWKATFNPPGPYWTHVLFTWKSKEGLKVYVNGTLSTSDPSGKVSHTYGEPHVNLVIGSEQDQTKRYENGAFDEFIIWERALTPDEIKMYFTAAIGKHSLLSSTPPAMPTAHSMVPTDAYHPIITNLTEERKCFQRPETVLRYLQNVSLSLPNKSLSEETALNLTQTFLRTVGEVLLLPSWTHVSEDNTMTLGLVDTIDTVMGHISANLQSREPHITLTGSSSMADFFVAKVLPPALSAPHYRFPAHGHSYIEIPREALHGKAWTTIVGLLYHSMHYYLNNIPPASTKIPEAVNCRDCLLSVASHLISLEVSPPPTLSQNLSGSPLITVHLRHKLTHKQYVDATNESNDLFLYCAFLNFSSGEGIWSSQGCALREGNLTHSVCHCTHLTNFAILMQVVPLQLTRGHQVALSSISYVGCSLSVLCLAATLVTFAVLSSVSTIRNQRYHIHANLSFAVLVAQVLLLISFRVEPGTVPCQVLAILLHYFFLSAFAWMLVEGLHLYSMVIKVFGSEDSKHLYYYGIGWGCPLLICIISISSSMDSYGTNDSCWLSLGSGAIWAFVGPALLVIVVNIVILVAVTRVISHVSTDNYKIHGDPSAFKLTAKAVAVLLPILGTSWVFGVLAVSDRALVFQYMFAVLNSLQGLFIFLFHCLLNSEVRAAFKHKTKVWSLTSSSARTTNTKPFSSDIVNGTRPGTASTKLSPWDKSSHSAHRVDLSAV

SEQ ID NO: 20 - protein, 2019 aa, Cricetulus griseus
MGAMTWLLPGIFLALLALNSEGAVLKKVIRHKRQSGLNMTLPEENQPVVFNHVYNIKLPVGSQCSVDLESVNGEKDLTPTPESSGTFQEHTVDGENQIVFTHRINIPRRACGCAAAPDVKELLSRLEELEMLVSSLREQCTMGTGCCLQPAEGRLDTRPFCSGRGNFSAEGCGCVCEPGWKGPNCSEPECPGNCNLQGQCLDGQCVCDEGFTGEDCSQLACPNDCNDQGRCVNGVCVCFEGYAGVDCGLEVCPVPCSEEHGTCLDGRCVCKDGFAGDDCNEPLCLNNCYNRGRCVENECVCDEGFTGEDCSELICPNDCFDRGRCVNGTCYCEEGFTGEDCGELTCPNNCQGHGQCEEGQCVCDEGFAGADCSEKRCPEDCHHRGRCLNGQCECDDGFMGADCGDLQCPNGCSGHGRCVNGQCVCDEGYTGEDCSQQRCPNDCHNRGLCVQGKCICEQGFKGFDCSEMSCPNDCHGHGRCVNGMCICDDEYTGEDCRDHRCPRDCSQRGRCLDGQCICEDGFTGPDCAELSCPNDCHGHGRCVNGQCICHEGFTGKDCKEQRCPSDCHGQGRCEDGQCICHEGFTGLDCGQRSCPNDCSNQGQCVSGRCICNEGYRGEDCSEVSPPKDLIVTEVTEETVNLAWDNEMRVTEYLIMYTPTHADGLEMQFRVPGDQTSTTIRELEPGVEYFIRVFAILENKRSIPVSARVATYLPAPEGLKFKSIKETSVEVEWDPLDIAFETWEIIFRNMNKEDEGEITKSLRRPETSYRQTGLAPGQEYEISLHIVKNNTRGPGLKKVTTTRLDAPSQIEVRDVTDTTALITWFKPLAEIDGIELSYGIKDVPGDRTTIDLTHEENQYSIGNLKPDTEYEVSLVSRRVDMASNPAKETFTTGLDAPRNLRRVSQTDNSITLEWRNVKADIDSYRIKYAPISGGDHAEVDVPKSQQATTKTTLTGLRPGTEYGVGVSAVKGDKESDPATINAATEIDAPRDFQVSETTQDSLTLLWKTPLAKFDRYRLNYSLPTGQSIEVQLPKDATSHVLTDLEPGKEYTVLLTAEKGRHKSKPARVKASTEQAPSLENLTVTEAGWDGLRLNWTADDLAYEYFVIQVQEANKVEAAHNFTVLGNLRATDIPGLKAATPYRVSIYGVARGSRTPVLSAEASTGKTPNLGEVSVAEVGWDALKLNWTAPEGVYQNFFIQVLEADTTQTVQNLTVPGGLRSVDLPGLRAATHYHITIRGVTQDFSTAPLSVEVLTEELPHLGGLSVTEVSWDGLILNWTTDDLAYEHFVIQVQEANNVEAAQNLTVPGSIRAMEIPGLKAATPYRVSIYGVIQGYRTPMLSADASTAKEPEIGNLNVSDVTPESFNLSWTATDGIFDMFTIEIIDSNRLLQMAEHNISGAERTAHISGLPPSTDFIVYLSGIAPSIRTKTISTTATTEAEPEVDNLLVSDATPGGFRLSWTADEGIFDSFVLRIRDTKKQSEPQEIILPSPERTRDITGLREATEYEIELYGISGGRRSQPVSAIATTAMGSPKEIMFSDITDNAATVSWKAPTAQVESFRITYVPMTGGAPSMVTVDGTDTETRLVKLTPGVEYHVSVIAMKGFEESDPVSGSLTTALDGPSGLLAANITDTEALALWQPAIATVDSYVISYTGERVPEVTRTVSGNTVEYELHDLEPATEYTLRIFAEKGHQKSSAITTKFTTDLDSPRDLTATEVQSETALLTWRPPRASITGYLLVYESLDGTVKEVIVGPDTTSYSLADLSPSTHYTVRIQALSGSLRSKLIQTIFTTIGLLYPFPRDCSQAMLNGDTTSGLYTIYINGDKTQALEVYCDMTSDGGGWIVFLRRKNGREDFYRNWKAYAAGFGDRREEFWLGLDNLSKITAQGQYELRVDLQDHGESAFAVYDRFSVGDAKSRYKLKVEGYSGTAGDSMNYHNGRSFSTYDKDTDSAITNCALSYKGAFWYKNCHRVNLMGRYGDNNHSQGVNWFHWKGHEYSIQFAEMKLRPSNFRNLEGRRKRA

SEQ ID NO: 21 - protein, 1466 aa, Cricetulus griseus
MMSFVQSGTWLLLALLHPTFIWAQQSSVDESGCSHLGQSYESRDVWKPEPCQICVCDSGSVLCDDIICDEDPLDCPNPEIPFGECCAICPQPSTAAPVPPDGHGPQGPKGDPGPPGIPGRNGDPGLPGQPGLPGPPGSPGICESCPTGGQNYSPQYDSYDVKSGVGAGGLGGGYPGPAGPPGPPGPPGSVGHPGSPGSPGYQGPPGEPGQAGPSGPPGPPGAIGPSGPAGKDGESGRPGRPGERGLPGPPGIKGPSGMPGFPGMKGHRGFDGRNGEKGETGAPGLKGENGLPGDNGAPGPMGPRGAPGERGRPGLPGAAGARGNDGARGSDGQPGPPGPPGTAGFPGSPGAKGEVGPAGSPGSNGSPGQRGEPGPQGHAGAQGPPGPPGNNGSPGGKGEMGPAGIPGAPGLMGARGPPGPAGTNGAPGQRGPSGEPGKNGAKGEPGARGERGEAGSPGIPGPKGEDGKDGSPGEPGANGLPGTAGERGAPGFRGPAGPNGIPGEKGPAGERGAPGPAGPRGVAGEPGRDGNPGGPGMRGVPGSPGGPGNDGKPGPPGSQGESGRPGPPGPSGPRGQPGVMGFPGPKGNDGAPGKNGERGGPGGPGLPGPAGKNGETGPQGPPGPTGPSGDKGESGPPGPQGLQGIPGTSGPPGENGKPGEPGPKGEVGAPGVPGGKGDAGAPGERGPPGTAGVPGLRGGAGPPGPEGGKGPAGPPGPPGTSGSPGLQGMPGERGGPGSPGPKGEKGEPGGAGADGAPGKDGPRGPTGPIGPPGPAGQPGDKGEGGAPGLPGIAGPRGGPGERGEHGPPGPAGFPGAPGQNGEPGAKGERGAPGEKGEGGPPGPAGLPGGSGPAGPPGPQGVKGERGSPGGPGAAGFPGGRGLPGPPGNNGNPGPPGPSGAPGKDGPPGPAGNSGSPGNPGVAGPKGDAGQPGEKGPPGAQGPPGSPGPLGIAGLTGARGLAGPPGMPGPRGSPGPQGIKGESGKPGASGHNGERGPPGPQGLPGQPGTAGEPGRDGNPGSDGQPGRDGSPGGKGDRGENGSPGAPGAPGHPGPPGPVGPAGKSGDRGETGPAGPSGAPGPAGSRGPPGPQGPRGDKGETGERGSNGIKGHRGFPGNPGPPGSPGAAGHQGAVGSPGPAGPRGPVGPHGPPGKDGTSGHPGPIGPPGPRGNRGERGSEGSPGHPGQPGPPGPPGAPGPCCGGGAAALGGGGEKSGGFSPYYGDEPMDFKINTEEIMSSLKSVNGQIESLISPDGSRKNPARNCRDLKFCHPDLKSGEYWVDPNQGCKMDAIKVFCNMETGETCINASPMTVPRKNWWTDASAEKKHVWFGESMNGGFQFSYGNPDLPEDVLDVQLAYLRLLSSRASQNITYHCKNSIAYMDQASGNVKKSLKLMGSNEGEFKAEGNSKFTYTVLEDGCAKHTGEWSKTVFEYRTRKALRLPIIDIAPYDIGGPDQEFGVDIGPVCFL

SEQ ID NO: 22 - protein, 221 aa, Cricetulus griseus
MAGKPVLHYFDGGGRMEPVRWLLAAAGVEFEEKFLKTRDDLARLRNDGSLMFQQVPMVEIDGMKLVQTRAILNYIASKYNLYGKDMKERALIDMYAEGIADLDEIVLHQPYIPQEEKEANLAKIKDKARNRYFPAYEKVLKGHGQDYLVGNRLSRADVYLVELLYHVEELDPSVLANFPLLKALRTRVSNLPTVKKFLQPGSQRKPYEDEKCVESAMKIFS

SEQ ID NO: 23 - protein, 607 aa, Cricetulus griseus
MWLRSLVKQLERGEASVVDLKKNLEYAATVLESVYIDETRRLLDTEDELSDIQSDAVPSEVRDWLASTFTRQMGMMLRRSDEKPRFKSIVHAVQAGIFVERMYRRTSNMVGLSYPPAVIDALKDVDKWSFDVFSLNDASGDHALKFIFYELLTRYDLISRFKIPISALVSFVEALEVGYSKHKNPYHNLMHAADVTQTVHYLLYKTGVANWLTELEIFAIIFSAAIHDYEHTGTTNNFHIQTRSDPAILYNDRSVLENHHLSAAYRLLQEDEEMNILVNLSKDDWREFRTLVIEMVMATDMSCHFQQIKAMKTALQQPEAIEKPKALSLMLHTADISHPAKAWDLHHRWTMSLLEEFFRQGDREAELGLPFSPLCDRKSTMVAQSQVGFIDFIVEPTFTVLTDMTEKIVSPLIDETSQTGGTGQRRSSLNNISASDAKRPGVKSSGSEGSAPINNSVIPVDYKSFKATWTEVVHVNRERWRAKVPKEEKAKKEAEEKARLAAEEKQKEMEAQDQTEQGKAEKKTSGETKGQVNGARTNKGKSPKGDKAGEKQQNGDLKEGKNKADKKDHSSTGNDSKKTDGTKKRSHDSPAPSTSSTSRITLPGDYG

SEQ ID NO: 24 - protein, 532 aa, Cricetulus griseus
MDRAGCLGAGLRGLCVAALVLVCAGHGARREDGGPACYGGFDLYFILDKSGSVLHHWNEIYYFVEQLAHRFISPQLRMSFIVFSTRGTTLMKLTEDREQIRQGLEELQKVLPGGDTYMHEGFERASEQIYYENSQGYRTASVIIALTDGELHEDLFFYSEREANRSRNLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFAVEDTYLLCPAPILKEVGMKAALQVSMNDGLSFISSSVIITTTHCSDGSILAIALLILFLLLALALLWWFWPLCCTVIIKEVPPPPVEESEEEDDDGLPKKKWPTVDASYYGGRGVGGIKRMEVRWGEKGSTEEGAKLEKAKNARVKMPEQEYEFPEPRNLNNNMRRPSSPRKWYSPIKGKLDALWVLLRKGYDRVSVMRPQPGDTGRCINFTRVKNTQAAKYPLRRFQRWLHCQDALNQIVSHGDQEESHFRERNAALDKK

SEQ ID NO: 25 - protein, 225 aa, Cricetulus griseus
MAFTSMSTEKSKCSGRVRHKLIVIGSPSTALAPGFYSGQAVSGLQKVVFVGLLGLLIASGFSYTINSNNESNVGGSGPQAVSINNQHNVANVDSNNGWGSWNALWDYENSFAATRIFAKKSCIVHKMNKNVMPSLQELDTLVKEQKDKEPEGATPKELMFSINPTRVEDLSTFGPKIAGMCQGIPTYVAEEIPGPNQPLYAQKCFTANILWIIKLSFCGTSEETY

SEQ ID NO: 26 - protein, 307 aa, Cricetulus griseus
MAMDQSTRDARHPSVTVRPADAALPAVAALLAETVRPADVESTEDAADPRVNVQDPEAALLSAPHSCFRCQLLYWVGALMLLLGVVCVPIALFTRIPTRPALTVTTSPTPGNSEKPSNLTVTPVPRNSCPKATGQGSSVFAILQAKEEVKLEPNRILKWHSQEGAGISKPLQGLRYDNVTNELVVNESGLYYVILQLKLRPVLKNTDRKVRGQVSLVLQLNPPIERPDNLALTVDLFPCSMETNLVEGSWSHLIPLKADDRLSVNLRAYLYGAQEAYKDWELSQTAITSFVLFLVPTDTPQELPSIR

SEQ ID NO: 27 - protein, 1026 aa, Cricetulus griseus
MTAIKMLQGSFPVFLLGALLGVLHAQQQEVISPDISTIDRNNNCPEKADCPVNVYFVLDTSESVAMQSPTDSLLYHMQQFVPQFISQLQNEFYLDQVALSWRYGGLHFSDQVEVFSPPGSDRASFTKSLQSIRSFRRGTFTDCALANMTQQIRQHVGRGVVNFAVVITDGHVTGNPCGGIKMQAERAREEGIRLFAVAPNRNLNEQGLRDIANTPHELYRNNYATMRPDSTEIDQDTINRIIKVMKHEAYGECYKVSCLEIPGPHGPKGYRGQKGAKGNMGEPGEPGQKGRQGDPGIEGPIGFPGPKGVPGFKGEKGEFGSDGRKGAPGLAGKNGTDGQKGKLGRIGPPGCKGDPGSRGPDGYPGEAGTPGEQGDQGAKGDSGRPGRRGPPGDPGDKGSKGYQGNNGAPGSPGVKGGKGGPGPRGPKGEPGRRGDPGTKGGPGTDGPKGEKGDPGPEGPRGLAGEVGSKGAKGDRGLPGPRGPQGALGELGKQGSRGDPGDAGPRGDSGQPGPKGDPGRPGFSYPGPRGTPGEKGEPGPPGPEGGRGDFGLKGAPGRKGDKGEPADPGPPGEPGPRGPRGIPGPEGEPGPPGDPGLTECDVMTYVRETCGCCDCEKRCGALDVVFVIDSSESIGYTNFTLEKNFVINVVNRLGAIAKDPKSETGTRVGVVQYSHEGTFEAIRLDDERVNSLSSFKEAVKNLEWIAGGTWTPSALKFAYNQLIKESRRQKTRVFAVVITDGRHDPRDDDLNLRALCDRDVTVTAIGIGDMFHETHESENLYSIACDKPQQVRNMTLFSDLVAEKFIDDMEDVLCPDPQIVCPELPCQTELYVAQCTQRPVDIVFLLDGSERLGEQNFHKARRFVEEVSRRLTLARKDDDPLNARMALLQYGSQNQQQVVFPLTYNLTTIHEALERTAYLNSFSHVGAGIVHAINNVVRGARGGARRHAELSFVFLTDGVTGNDSLEESVHSMRKQNVVPTVVAVGSDVDMDVLTKISLGDRAAIFREKDFDSLAQPSFFDRFIRWIC

SEQ ID NO: 28 - DNA, 17 bp, synthetic construct
  misc_feature 1..17: note = Probe for qPCR
  source 1..17: mol_type = other DNA
caatctccaccagcgac

SEQ ID NO: 29 - DNA, 20 bp, synthetic construct
  misc_feature 1..20: note = Forward Primer for qPCR
  source 1..20: mol_type = other DNA
gacccctgcatggtctttga

SEQ ID NO: 30 - DNA, 22 bp, synthetic construct
  misc_feature 1..22: note = Reverse Primer for qPCR
  source 1..22: mol_type = other DNA
accgtaatgctgactcccaagt

(19) United States
(12) Patent Application Publication
(10) Pub. No.: US 2023/0250401 A1
(43) Pub. Date: Aug. 10, 2023

(54) METHOD FOR SELECTING CELL

(71) Applicant: KYOWA KIRIN CO., LTD., Tokyo (JP)

(72) Inventors: Koichi YAMAMOTO, Tokyo (JP); Yuichi MATSUMOTO, Tokyo (JP); Keina YAMAGUCHI, Tokyo (JP); Hiroto FUJII, Tokyo (JP); Toshiyuki SUZAWA, Tokyo (JP)

(73) Assignee: KYOWA KIRIN CO., LTD., Tokyo (JP)

(21) Appl. No.: 18/300,892

(22) Filed: Apr. 14, 2023

Related U.S. Application Data
(62) Division of application No. 16/474,164, filed on Jun. 27, 2019.

(30) Foreign Application Priority Data
Dec. 28, 2016 (JP) 2016-256279

(51) Int. Cl. C12N 5/16; C07K 16/46; C12N 15/09; C12Q 1/04; C12Q 1/68; C12N 15/113; C12Q 1/6881

(52) U.S. Cl. CPC C12N 5/16; C07K 16/46; C12N 15/09; C12Q 1/04; C12Q 1/68; C12N 15/113; C12Q 1/6881

(57) ABSTRACT

A method for selecting a cell strain in which reduction of a recombinant protein is suppressed, and uses of the selected cell are disclosed. The method for selecting a cell, includes a first step: measuring the expression level of a gene in a cell, the gene being at least one gene selected from genes comprising any one of the base sequences represented by SEQ ID NOS: 1 to 16 or orthologous genes thereof; and a second step: comparing the expression level of the gene measured in the first step with a control value of the expression level of the gene in a control cell, and evaluating the expression level capable of suppressing reduction of a recombinant protein based on a difference therebetween.

### CROSS-REFERENCE

This application is a Divisional of U.S. application Ser. No. 16/474,164, filed on Jun. 27, 2019, which is a National Stage of International Application No. PCT/JP2017/046939, filed on Dec. 27, 2017, which claims priority from Japanese Patent Application No. 2016-256279, filed on Dec. 28, 2016, the contents of all of which are incorporated herein by reference in their entireties.

### SEQUENCE LISTING

The instant application contains a Sequence Listing which has been filed electronically in xml format and is hereby incorporated by reference in its entirety. Said xml copy, created on Apr. 14, 2023, is named Q286702_Sequence_Listing_as_filed and is 61,734 bytes in size.

### TECHNICAL FIELD

The present invention relates to a method for selecting a cell, and more specifically relates to a method for selecting a cell capable of suppressing reduction of a recombinant protein.

### Background Art

With the recent development of gene recombination techniques, biopharmaceutical products such as antibodies have begun to be widely supplied. These biopharmaceutical products are produced by using a production cell prepared by introducing an expression vector containing a base sequence encoding a recombinant protein (hereinafter sometimes also referred to as “target protein” for distinguishing it from a protein translated based on an endogenous gene and secreted from the same cell) into a host cell such as E. coli, yeast, an insect cell, a plant cell, or an animal cell.

In a step generally used as a step of producing biologics (protein drugs), at first, production cells are cultured under appropriate conditions, and allowed to secrete a target protein in a culture broth. The culture broth containing the target protein is subjected to purification after removing the production cells which are no longer needed.

However, in the culture broth before purification, many proteins having various enzyme activities derived from the production cells are present other than the target protein. Thus, due to the activities of those proteins, the target protein may be sometimes degraded or denatured. Further, even if the culture broth containing the target protein is purified, depending on the purification degree, proteins derived from the production cells remain, and therefore, the target protein may be sometimes degraded or denatured.

As one of the causes of degrading or denaturing the target protein, a reducing activity of thioredoxin reductase or the like endogenously present in the production cells is known. Specifically, a disulfide bond contained in the protein is reduced by thioredoxin reductase, and therefore, a high-order structure necessary for expressing the activity of the protein is lost (Non-Patent Document 1).

In order to suppress this reducing activity causing degradation or denaturation of the target protein, an attempt to add a thioredoxin reductase inhibitor or an oxidizing agent to the culture broth, or to incorporate air sparging or the like to the culture broth in the production step has been made (Patent Document 1, and Non-Patent Documents 2 and 3).

Further, for the purpose of establishing production cells in which a reducing activity is suppressed, an attempt to produce a protein using cells in which the thioredoxin reductase gene is knocked down has been made. However, the establishment of production cells focusing on such gene has not yet been put into practical use because there is a problem that the growth of the production cells is significantly deteriorated due to the knockdown, or the like (Non-Patent Document 4).

Further, for selecting production cells in which a reducing activity is suppressed, a method other than evaluation of a reducing activity imitating the production processes (Non-Patent Document 1) has not been known at present, and it is not easy to evaluate and select a large number of cells.

As described above, for suppressing the reducing activity causing degradation or denaturation of the target protein, many methods targeted at culture or a step after that during the production have been proposed. However, at present, only a few methods focus on a step of establishing production cells, such as improvement or selection of a host cell or a genetically modified cell or the like.

### CITATION LIST

### Patent Literature

Patent Document 1: JP-A-2014-129358

### Non Patent Literature

Non-Patent Document 1: Biotechnology and Bioengineering, 622-632, 107(4), 2010

Non-Patent Document 2: Biotechnology and Bioengineering, 452-461, 106(3), 2010

Non-Patent Document 3: Biotechnology and Bioengineering, 734-742, 112(4), 2015

Non-Patent Document 4: Journal of Biotechnology, 261-267, 157, 2012

### SUMMARY OF INVENTION

### Problem to be Solved by the Invention

As described above, in order to produce a protein using a recombinant host cell, a cell in which degradation or denaturation of a target protein does not occur, and above all, particularly, a cell in which reduction of a target protein does not occur is needed for a use in the production.

Therefore, an object of the present invention is to provide a method for selecting a cell in which a reducing activity against a recombinant protein (target protein) is suppressed.

### Means for Solving the Problems

The present inventors made intensive studies for achieving the above object, and as a result, they surprisingly found that the strength of the reducing activity causing degradation or denaturation of a target protein varies in each cell. Then, they specified a gene group whose association with this reducing activity has totally not been suggested, and found a correlation between the expression level of the gene and the reducing activity of cells. Further, they found that by selecting a cell using the expression of the gene or the expression level of a protein encoded by the gene as an index, when the target protein is produced using the cell, the reducing activity for the target protein can be suppressed, and thus completed the present invention.

That is, the present invention relates to the following [1] to [15].

[1] A method for selecting a cell, comprising the following first step and second step:

a first step: measuring the expression level of a gene in a cell, the gene being at least one gene selected from genes comprising any one of the base sequences represented by SEQ ID NOS: 1 to 16 or orthologous genes thereof; and

a second step: comparing the expression level of the gene measured in the first step with a control value of the expression level of the gene in a control cell or comparing the expression level of the gene in the respective cells, and evaluating the expression level capable of suppressing reduction of a recombinant protein based on a difference therebetween.

[2] A method for selecting a cell, comprising the following first step and second step:

a first step: measuring the expression level of a protein encoded by at least one gene selected from genes comprising any one of the base sequences represented by SEQ ID NOS: 1 to 16 or orthologous genes thereof; and

a second step: comparing the expression level of the protein encoded by the gene measured in the first step with a control value of the expression level of the protein encoded by the gene in a control cell or comparing the expression level of the protein encoded by the gene in the respective cells, and evaluating the expression level capable of suppressing reduction of a recombinant protein based on a difference therebetween.

[3] A method for selecting a cell, comprising the following first step and second step:

a first step: measuring the expression level of a gene in a cell, the gene being at least one gene selected from genes comprising any one of the base sequences represented by SEQ ID NOS: 1 to 16 or orthologous genes thereof; and

a second step: calculating and comparing a relative expression level and selecting a cell according to the following procedures (a) to (d);

(a) calculating a relative quantitative value by dividing the expression level of the gene in a test cell measured in the first step by the expression level of a standard gene in the test cell

a relative quantitative value=(the expression level of the gene in a test cell measured in the first step) / (the expression level of a standard gene),

(b) calculating a control value by dividing the expression level of the gene in a control cell by the expression level of the standard gene in the control cell

a control value=(the expression level of the gene in a control cell)/(the expression level of the standard gene),

(c) calculating a relative expression level by dividing the relative quantitative value for the test cell calculated in (a) by the control value calculated in (b)

a relative expression level=[the relative quantitative value for the test cell calculated in (a)]/[the control value calculated in (b)], and

(d) comparing the relative quantitative values calculated in (a) or the relative expression level calculated in (c) among the respective test cells, and thereby selecting a cell in which each value is high or low.

[4] The method according to [3], wherein in the second step (d), the cell in which the relative quantitative value or the relative expression level is high is a cell in which the relative quantitative value or the relative expression level is twice or more the control value, or the cell in which the relative quantitative value or the relative expression level is low is a cell in which the relative quantitative value or the relative expression level is ½ or less of the control value.
[5] The method according to any one of [1] to [4], comprising introducing at least one gene selected from genes comprising any one of the base sequences represented by SEQ ID NOS: 1 to 9 or orthologous genes thereof into the cell, thereby transforming the cell before the first step.
[6] The method according to any one of [1] to [5], comprising knocking down or knocking out at least one gene selected from genes comprising any one of the base sequences represented by SEQ ID NOS: 10 to 16 or orthologous genes thereof in a cell before the first step.
[7] The method according to any one of [1] to [6], comprising introducing a gene encoding a recombinant protein into the cell, thereby transforming the cell before the first step.
[8] A method for obtaining a cell producing a recombinant protein, comprising selecting a cell by the method according to any one of [1] to [7].
[9] A method for producing a recombinant protein using a cell selected or obtained by the method according to any one of [1] to [8].
[10] The method according to any one of [1] to [9], wherein the cell is a cell derived from a mammalian cell.
[11] The method according to any one of [1] to [10], wherein the gene is Plet1 gene.
[12] The method according to any one of [1] to [11], wherein the recombinant protein is an antibody.
[13] A cell capable of suppressing reduction of a recombinant protein, wherein at least one gene selected from genes comprising any one of the base sequences represented by SEQ ID NOS: 1 to 9 or orthologous genes thereof is introduced to transform the cell.
[14] A cell capable of suppressing reduction of a recombinant protein, wherein at least one gene selected from genes comprising any one of the base sequences represented by SEQ ID NOS: 10 to 16 or orthologous genes thereof is knocked down or knocked out.
[15] A method for increasing probability of obtaining a cell capable of suppressing reduction of a recombinant protein by using at least one gene selected from genes comprising any one of the base sequences represented by SEQ ID NOS: 1 to 16 or orthologous genes thereof as an index.

### Effects of the Invention

According to the method of the present invention, from a cell population having been subjected to gene recombination, a cell in which a reducing activity against a target protein is suppressed can be easily selected with high probability. By combining the method with evaluation of an index other than susceptibility to reduction, such as productivity of a protein or the like, a gene recombinant protein-producing cell having a desired property for producing a pharmaceutical product can be efficiently selected.

Further, according to the method of the present invention, a cell in which a reducing activity against a target protein is suppressed can be obtained. By using this cell, a target protein-producing cell in which a reducing activity against the target protein is suppressed can be obtained.

### BRIEF DESCRIPTION OF DRAWINGS

FIG. 1 is a figure showing the results of capillary electrophoresis showing that an antibody produced by A #1 strain is not reduced and is stably present as the whole antibody, and that an antibody produced by A #2 strain is degraded over time by reduction.

FIG. 2 is a figure showing changes in the relative expression level of Plet1 gene in 80 days of subculture period of antibody C-producing cells.

### EMBODIMENTS FOR CARRYING OUT THE INVENTION

A first embodiment of the present invention is a method for selecting a cell in which a reducing activity for a recombinant protein (target protein) is suppressed, comprising the following first step and second step:

a first step: measuring the expression level of a gene in a cell, the gene being at least one gene selected from genes comprising any one of the base sequences represented by SEQ ID NOS: 1 to 16 or orthologous genes thereof; and

a second step: comparing the expression level of the gene measured in the first step with a control value of the expression level of the gene, and evaluating the expression level capable of suppressing reduction of a recombinant protein based on a difference therebetween.

The present inventors compared, in cells producing an antibody, a cell in which the antibody is not easily reduced and a cell in which the antibody is easily reduced through a gene expression difference analysis using a microarray, and found that the expression level of genes comprising a base sequence represented by any of SEQ ID NOS: 1 to 16 in the cell is greatly different between the cell in which the antibody is not easily reduced and the cell in which the antibody is easily reduced.

The base sequences represented by SEQ ID NOS: 1 to 16 are base sequences registered in Affimetrix or NCBI (National Center for Biotechnology Information), and a gene comprising any one of these or an orthologous gene thereof can be obtained by searching database based on the gene name or a partial sequence from the web page of Affimetrix (www.affymetrix.com/analysis/netaffx/xmlquery_ex.affx?netaffx=wtgene_transcrip) or the web page of NCBI (www.ncbi.nlm.nih.gov). On the web page of Affimetrix, it is registered as ID: 18083239. Incidentally, the “orthologous gene” refers to an analogous gene encoding a protein having a homologous function present in a different organism.

The base sequences represented by SEQ ID NOS: 1, 4, 7, 8, and 10 to 16 are base sequences of Plet1 gene, matrilin 4 gene, G-protein-coupled receptor 133 gene, tenascin C gene, collagen alpha-1(III) chain gene, glutathione S-transferase alpha-3 gene, calcium/calmodulin-dependent 3′,5′-cyclic nucleotide phosphodiesterase 1C gene, anthrax toxin receptor 1 gene, gastrokine-1 gene, tumor necrosis factor ligand superfamily member 9 gene, and collagen, type VI, α2 gene, respectively.

The “Plet1 gene” means a gene encoding Plet1. Plet1 is placenta-expressed transcript 1 protein expressed in placenta and is a protein comprising an amino acid sequence represented by SEQ ID NO: 17. Plet1 is present in various types of mammals, and a gene encoding Plet1 derived from any of various mammals can be used, however, it is preferred to use a gene of a mammal from which a cell to be used as a host cell is derived. As the Plet1 gene, for example, Plet1 gene of a Chinese hamster comprising a base sequence represented by SEQ ID NO: 1 is exemplified.

The “matrilin 4 gene” means a gene encoding matrilin 4 (Matn4). Matrilin 4 is a protein comprising an amino acid sequence represented by SEQ ID NO: 18. Matrilin 4 is present in various types of mammals, and a gene encoding matrilin 4 derived from any of various mammals can be used, however, it is preferred to use a gene of a mammal from which a cell to be used as a host cell is derived. As the matrilin 4 gene, for example, matrilin 4 gene of a Chinese hamster comprising a base sequence represented by SEQ ID NO: 4 is exemplified.

The “G-protein-coupled receptor 133 gene” means a gene encoding G-protein-coupled receptor 133. The G-protein-coupled receptor 133 gene is a protein comprising an amino acid sequence represented by SEQ ID NO: 19. The G-protein-coupled receptor 133 gene is present in various types of mammals, and a gene encoding G-protein-coupled receptor 133 gene derived from any of various mammals can be used, however, it is preferred to use a gene of a mammal from which a cell to be used as a host cell is derived. As the G-protein-coupled receptor 133 gene, for example, G-protein-coupled receptor 133 gene of a Chinese hamster comprising a base sequence represented by SEQ ID NO: 7 is exemplified.

The “tenascin C gene” means a gene encoding tenascin C. Tenascin C is a protein comprising an amino acid sequence represented by SEQ ID NO: 20. Tenascin C is present in various types of mammals, and a gene encoding tenascin C derived from any of various mammals can be used, however, it is preferred to use a gene of a mammal from which a cell to be used as a host cell is derived. As the tenascin C gene, for example, tenascin C gene of a Chinese hamster comprising a base sequence represented by SEQ ID NO: 8 is exemplified.

The “collagen alpha-1(III) chain gene” means a gene encoding collagen alpha-1(III) chain. Collagen alpha-1(III) chain is a protein comprising an amino acid sequence represented by SEQ ID NO: 21. Collagen alpha-1(III) is present in various types of mammals, and a gene encoding collagen alpha-1(III) derived from any of various mammals can be used, however, it is preferred to use a gene of a mammal from which a cell to be used as a host cell is derived. As the collagen alpha-1(III) chain gene, for example, collagen alpha-1(III) chain gene of a Chinese hamster comprising a base sequence represented by SEQ ID NO: 10 is exemplified.

The “glutathione S-transferase alpha-3 gene” means a gene encoding glutathione S-transferase alpha-3. Glutathione S-transferase alpha-3 is a protein comprising an amino acid sequence represented by SEQ ID NO: 22. Glutathione S-transferase alpha-3 is present in various types of mammals, and a gene encoding glutathione S-transferase alpha-3 derived from any of various mammals can be used, however, it is preferred to use a gene of a mammal from which a cell to be used as a host cell is derived. As the glutathione S-transferase alpha-3 gene, for example, glutathione S-transferase alpha-3 gene of a Chinese hamster comprising a base sequence represented by SEQ ID NO: 11 is exemplified.

The “calcium/calmodulin-dependent 3′,5′-cyclic nucleotide phosphodiesterase 1C gene” means a gene encoding calcium/calmodulin-dependent 3′,5′-cyclic nucleotide phosphodiesterase 1C. Calcium/calmodulin-dependent 3′,5′-cyclic nucleotide phosphodiesterase 1C is a protein comprising an amino acid sequence represented by SEQ ID NO: 23. Calcium/calmodulin-dependent 3′,5′-cyclic nucleotide phosphodiesterase 1C is present in various types of mammals, and a gene encoding calcium/calmodulin-dependent 3′,5′-cyclic nucleotide phosphodiesterase 1C derived from any of various mammals can be used, however, it is preferred to use a gene of a mammal from which a cell to be used as a host cell is derived. As the calcium/calmodulin-dependent 3′,5′-cyclic nucleotide phosphodiesterase 1C gene, for example, calcium/calmodulin-dependent 3′,5′-cyclic nucleotide phosphodiesterase 1C gene of a Chinese hamster comprising a base sequence represented by SEQ ID NO: 12 is exemplified.

The “anthrax toxin receptor 1 gene” means a gene encoding anthrax toxin receptor 1. Anthrax toxin receptor 1 is a protein comprising an amino acid sequence represented by SEQ ID NO: 24. Anthrax toxin receptor 1 is present in various types of mammals, and a gene encoding anthrax toxin receptor 1 derived from any of various mammals can be used, however, it is preferred to use a gene of a mammal from which a cell to be used as a host cell is derived. As the anthrax toxin receptor 1 gene, for example, anthrax toxin receptor 1 gene of a Chinese hamster comprising a base sequence represented by SEQ ID NO: 13 is exemplified.

The “gastrokine-1 gene” means a gene encoding gastrokine-1. Gastrokine-1 is a protein comprising an amino acid sequence represented by SEQ ID NO: 25. Gastrokine-1 is present in various types of mammals, and a gene encoding gastrokine-1 derived from any of various mammals can be used, however, it is preferred to use a gene of a mammal from which a cell to be used as a host cell is derived. As the gastrokine-1 gene, for example, gastrokine-1 gene of a Chinese hamster comprising a base sequence represented by SEQ ID NO: 14 is exemplified.

The “tumor necrosis factor ligand superfamily member 9 gene” means a gene encoding tumor necrosis factor ligand superfamily member 9. Tumor necrosis factor ligand superfamily member 9 is a protein comprising an amino acid sequence represented by SEQ ID NO: 26. Tumor necrosis factor ligand superfamily member 9 is present in various types of mammals, and a gene encoding tumor necrosis factor ligand superfamily member 9 derived from any of various mammals can be used, however, it is preferred to use a gene of a mammal from which a cell to be used as a host cell is derived. As the tumor necrosis factor ligand superfamily member 9 gene, for example, tumor necrosis factor ligand superfamily member 9 gene of a Chinese hamster comprising a base sequence represented by SEQ ID NO: 15 is exemplified.

The “collagen, type VI, α2 gene” means a gene encoding collagen, type VI, alpha 2. Collagen, type VI, α2 is a protein comprising an amino acid sequence represented by SEQ ID NO: 27. Collagen, type VI, α2 is present in various types of mammals, and a gene encoding collagen, type VI, α2 derived from any of various mammals can be used, however, it is preferred to use a gene of a mammal from which a cell to be used as a host cell is derived. As the collagen, type VI, α2 gene, for example, collagen, type VI, α2 gene of a Chinese hamster comprising a base sequence represented by SEQ ID NO: 16 is exemplified.

Among the above-mentioned genes, in the present invention, it is preferred to evaluate the ability of the cell capable of suppressing reduction of a recombinant protein using a difference in the expression level of Plet1 [Placenta expressed transcrript 1 protein (ID: 18083239)] gene as an index. Incidentally, in this description, the “ID” denotes a registration number on the web page of Affimetrix, Inc. (www.affymetrix.com/analysis/netaffx/xmlquery_ex.affx?netaffx=wtgene_transcript).

Hereinafter, in this description, the present invention will be described mainly using Plet1 gene among the above-mentioned genes, however, the invention is not limited to using Plet1 gene, and any of the above-mentioned genes can be used. Further, as listed in Table 2, a gene comprising any one of the base sequences represented by SEQ ID NOS: 2, 3, 5, 6, and 9 whose ID corresponds to 17952793, 17952795, 17957314, 17958207, and 18024962, respectively, can also be used.

In the present invention, the degradation or denaturation of a protein means that the structure of the protein is chemically or enzymatically affected, and a covalent bond or a non-covalent bond is cleaved so that the protein is separated into subunits, protein fragments, polypeptide fragments, amino acids or the like, which constitutes the protein, or that the secondary structure, the tertiary structure, or the quaternary structure of the protein is changed from the naturally occurring state, or that association, aggregation, or dissociation occurs.

In the present invention, the reduction is one of the causes of degradation or denaturation of a protein, and means, for example, that a disulfide bond (—S—S— bond) crosslinking in a molecule or between molecules of the protein is dissociated at a plurality or one or more sites, and a free sulfhydryl group (—SH group) occurs at a plurality or one or more sites.

The reduction of a protein occurs, for example, as follows. An oxidoreductase that is present in a cell is released or the like from the cell and reduces the protein. Examples of the oxidoreductase include dehydrogenases, cytochromes, catalases, oxidases, oxygenases, and fatty acid desaturases. Among these, thioredoxin reductase is considered to be greatly involved in degradation of a protein by reduction.

The present invention can be particularly applied to a protein having a disulfide bond in a molecule or between molecules of the protein. The disulfide bond may be present at one or more sites in a molecule or between molecules of the protein. Further, in the case of an intermolecular disulfide bond, proteins to be bonded may be the same or different.

Examples of the protein having a disulfide bond in a molecule or between molecules include antibodies (for example, IgG1 to 4, IgM, IgE, IgD, and IgA), single-chain antibodies, Fab, and F(ab′)₂ and the like.

In the present invention, the suppression of reduction or a reducing activity refers to suppression of the above-described reduction of a protein. In particular, in the case of a recombinant protein, it refers that a recombinant protein produced by a cell is maintained in the native form, for example, in a state where a disulfide bond is formed at a proper site.

In the present invention, the ease of reduction of a protein can be determined by homogenizing cells producing a target protein, collecting a solution over time from a cell homogenate incubated under an anaerobic condition, and subsequently, measuring the molecular weight of the produced protein contained in the collected homogenate solution by electrophoresis, capillary electrophoresis, gel filtration HPLC, peptide mapping, or the like. That is, in the case where the produced protein is easily decomposed, the molecular weight of the protein is decreased, and therefore, this is measured by a physicochemical method such as electrophoresis and can be used as an index of the ease of reduction.

Incidentally, even if a protein having a conformation like an antibody is reduced, the conformation is maintained, and therefore, degradation by reduction cannot be detected by an analysis method such as ordinary gel filtration HPLC. In such a case, the reduction can be detected by performing a treatment such as alkylation of a free thiol group formed by reduction so as to denature the protein, and then, performing the above-mentioned analysis.

The cell to be used in the present invention is not particularly limited as long as it is a cell having an ability to express a recombinant protein. Examples thereof include cells derived from mammals that are generally used for producing a recombinant protein such as Chinese hamster ovary cells (CHO), baby hamster kidney cells (BHK), human cells (HT1080 fibrosarcoma cells, Per.C6), mouse myeloma cells (NS0, SP2/0), and Madin-Darby canine kidney cell-derived cells (MDCK), but the cell is not limited thereto, and a cell derived from an animal such as a human, a mouse, a rat, a hamster, a guinea pig, a rabbit, a dog, cattle, a horse, sheep, a monkey, or a pig may be used. Further, as the CHO cells, a substrain such as a CHO-K1 strain, a CHO-DG44 strain, a CHO-S strain, or a DUKX-B11 strain may be used.

In the present invention, the “recombinant protein” means a protein to be obtained by inserting a gene encoding the recombinant protein into an appropriate expression vector using a recombinant DNA technique and transforming a cell using this vector. Examples of the recombinant protein to be used in the present invention include antibodies, and various peptides and proteins having already been used or developed as pharmaceutical products, and cytokines.

As a medium for culturing the cell or a cell obtained by transformation, a medium generally used for culturing a cell as described above or a cell obtained by transformation is used. Examples of such a medium include IMDM, MEM, DMEM, RPMI-1640, X-VIVO15 medium, EX-CELL series media (SAFC Biosciences), BalanCD CHO series media (JX), other commercially available media and custom-made media developed for animal cells.

To the medium, fetal bovine serum may be added, and, a serum-free medium can also be used. When fetal bovine serum is added, the concentration thereof is preferably from about 5 to 20%. Further, in the medium, various components such as amino acids, vitamins, saccharides, soybean hydrolysate, yeast extract, and trace metals can be used by being mixed at an appropriate ratio.

In the first embodiment, in the first step, the expression level of the gene (at least one gene selected from genes comprising any one of the base sequences represented by SEQ ID NOS: 1 to 16 or orthologous genes thereof) in a test cell is measured. The gene whose expression level is measured is preferably Plet1.

A method for measuring the expression level of the gene is not particularly limited, and, for example, a method such as quantitative real-time PCR (qPCR), a reverse transcription quantitative real-time PCR (RT-qPCR) method, an expression difference analysis by RNA-seq using NGS, an expression difference analysis using a DNA microarray, Northern blotting, or ELISA (Enzyme-linked Immunosorbent Assay) is exemplified. Among these, the RT-qPCR method can perform rapid measurement, and therefore is preferred. The RT-qPCR method will be described in detail in the section of Examples.

In the first embodiment, in the second step, the expression level of the gene measured in the first step is (i) compared with a control value of the expression level of the gene in a control cell or (ii) compared with the expression level of the gene in respective cells, and the expression level capable of suppressing reduction of a recombinant protein is evaluated based on a difference therebetween.

The evaluation of the expression level capable of suppressing reduction of a recombinant protein in the second step of the first embodiment is specifically preferably performed according to the following procedures (a) to (d):

(a) calculating a quantitative value by dividing the expression level of the gene in a test cell measured in the first step by the expression level of a standard gene in the test cell,

a relative quantitative value=(the expression level of the gene in a test cell measured in the first step)/(the expression level of a standard gene)

(b) calculating a control value by dividing the expression level of the gene in a control cell by the expression level of the standard gene in the control cell,

a control value=(the expression level of the gene in a control cell)/(the expression level of the standard gene)

(c) calculating a relative expression level by dividing the relative quantitative value for the test cell calculated in (a) by the control value calculated in (b), and

a relative expression level=[the relative quantitative value for the test cell calculated in (a)]/[the control value calculated in (b)]

(d) comparing the relative quantitative value calculated in (a) or the relative expression level calculated in (c) among the respective test cells, and selecting a cell in which each value is high or low.

The procedure (d) is more preferably performed according to the following procedure (d-1) or (d-2):

(d-1) comparing the relative quantitative values calculated in (a) among the respective test cells, and thereby selecting a cell in which the value is high (when at least one gene selected from genes comprising any one of the base sequences represented by SEQ ID NOS: 1 to 9 or orthologous genes thereof is used) or low (when at least one gene selected from genes comprising any one of the base sequences represented by SEQ ID NOS: 10 to 16 or orthologous genes thereof is used), or

(d-2) comparing the relative expression level calculated in (c) among the respective test cells, and thereby selecting a cell in which the value is high (when at least one gene selected from genes comprising any one of the base sequences represented by SEQ ID NOS: 1 to 9 or orthologous genes thereof is used) or low (when at least one gene selected from genes comprising any one of the base sequences represented by SEQ ID NOS: 10 to 16 or orthologous genes thereof is used).

As the cell in which the relative quantitative value is high, a cell in which the relative quantitative value is twice or more, for example, 5 times or more, 7 times or more, 10 times or more, 20 times or more, 25 times or more, 30 times or more, 50 times or more, or 100 times or more the relative quantitative value (also referred to as “control value”) of a cell (control cell) showing the smallest value among the test cells is exemplified.

In particular, as the cell in which the relative quantitative value is high, a cell in which the relative quantitative value is preferably 5 times or more, more preferably 10 times or more, further more preferably 20 times or more, still further more preferably 25 times or more, yet still further more preferably 30 times or more, 50 times or more, and most preferably 100 times or more the control value is exemplified.

As the cell in which the relative quantitative value is low, a cell in which the relative quantitative value is ½ or less, preferably ⅕ or less, more preferably 1/10 or less, further more preferably 1/20 or less of the relative quantitative value (also referred to as “control value”) of a cell (control cell) showing the largest value among the test cells is exemplified.

As the cell in which the relative expression level is high, a cell in which the relative expression level is twice or more, for example, 5 times or more, 7 times or more, 10 times or more, 20 times or more, 25 times or more, 30 times or more, 50 times or more, or 100 times or more the relative expression level of a cell showing the smallest value among the test cells is exemplified.

In particular, as the cell in which the relative expression level is high, a cell in which the relative expression level is preferably 5 times or more, more preferably 10 times or more, further more preferably 20 times or more, still further more preferably 25 times or more, yet still further more preferably 30 times or more, even yet still further more preferably 50 times or more, and most preferably 100 times or more the relative expression level of a cell showing the smallest value among the test cells is exemplified.

As the cell in which the relative expression level is low, a cell in which the relative expression level is ½ or less, preferably ⅕ or less, more preferably 1/10 or less, further more preferably 1/20 or less of the relative expression level of a cell showing the largest value among the test cells is exemplified.

Further, the evaluation of the expression level capable of suppressing reduction of a recombinant protein in the second step of the first embodiment can also be performed according to the following procedures (e) to (g):

(e) calculating a relative quantitative value by dividing the expression level of the gene in a test cell measured in the first step by the expression level of a standard gene in the test cell, a relative quantitative value=(the expression level of the gene in a test cell measured in the first step)/(the expression level of a standard gene)

(f) comparing a relative quantitative value for the test cell calculated in (e) with a relative quantitative value calculated in the same manner for another cell.

(g) comparing the relative quantitative value calculated in (e) among the respective test cells, and thereby selecting a cell in which the value is high (when at least one gene selected from genes comprising any one of the base sequences represented by SEQ ID NOS: 1 to 9 or orthologous genes thereof is used) or low (when at least one gene selected from genes comprising any one of the base sequences represented by SEQ ID NOS: 10 to 16 or orthologous genes thereof is used).

As the standard gene, any gene can be used without particular limitation as long as it is a gene generally expressed in a cell, and examples thereof include GAPDH (Glyceraldehyde-3-phosphate dehydrogenase), β-actin, β2-microglobulin, HPRT1 (Hypoxanthine phosphoribosyltransferase 1), globulin, ubiquitin and the like. Further, the measurement of the expression level of the standard gene can be performed by the RT-qPCR method or the like in the same manner as the expression level of the above-mentioned gene [Genome Biol., 1-11, 3(7), 2002]. Preferably, GAPDH is used.

As the control cell, for example, a cell in which a recombinant protein is easily reduced is exemplified. As the control cell, a sample, which is derived from the same cell as the cell measured for the expression level of the gene in the first step, but whose sampling time is different, may be used, or it may be a cell derived from a different cell. Further, as the control cell, one may be selected from the test cells, and in that case, any cell may be selected among the respective test cells.

Specifically, for example, in the case where the evaluation is performed using at least one gene selected from genes comprising any one of the base sequences represented by SEQ ID NOS: 1 to 9 or orthologous genes thereof, when a cell showing the lowest relative quantitative value among the values of the respective test cells subjected to measurement is used as the control cell, the difference from that of the test cell to be selected becomes larger, and therefore, the analysis of the results is easier.

On the other hand, in the case where the evaluation is performed using at least one gene selected from genes comprising any one of the base sequences represented by SEQ ID NOS: 10 to 16 or orthologous genes thereof, when a cell showing the highest relative quantitative value is used as the control cell, the difference from that of the test cell to be selected becomes larger, and therefore, the analysis of the results is easier.

That is, the control cell is preferably a cell showing the lowest relative quantitative value when at least one gene selected from genes comprising any one of the base sequences represented by SEQ ID NOS: 1 to 9 or orthologous genes thereof is used, and is a cell showing the highest relative quantitative value when at least one gene selected from genes comprising any one of the base sequences represented by SEQ ID NOS: 10 to 16 or orthologous genes thereof is used.

A second embodiment of the present invention is a method for selecting a cell in which a reducing activity for a recombinant protein is suppressed, comprising the following first step and second step:

a first step: measuring the expression level of a protein encoded by at least one gene selected from genes comprising any one of the base sequences represented by SEQ ID NOS: 1 to 16 or orthologous genes thereof; and

a second step: comparing the expression level of the protein encoded by the gene measured in the first step with a control value of the expression level of the protein encoded by the gene in a control cell or comparing the expression level of the protein encoded by the gene in the respective cells, and evaluating the expression level capable of suppressing reduction of a recombinant protein based on a difference therebetween.

In the second embodiment, in the first step, the expression level of a protein encoded by the gene (at least one gene selected from genes comprising any one of the base sequences represented by SEQ ID NOS: 1 to 16 or orthologous genes thereof) expressed in a test cell is measured. The protein whose expression level is measured is preferably Plet1.

A method for measuring the expression level of the protein is not particularly limited, and, for example, a method such as ELISA, Western blotting, FACS (Fluorescence-activated Cell Sorting), HPLC (High Performance Liquid Chromatography), or LC-MS is exemplified. Among these, the ELISA method can perform rapid measurement, and therefore is preferred.

In the second embodiment, in the second step, the expression level of a protein encoded by the gene (at least one gene selected from genes comprising any one of the base sequences represented by SEQ ID NOS: 1 to 16 or orthologous genes thereof) measured in the first step is (i) compared with a control value of the expression level of a protein encoded by the gene in a control cell or (ii) compared with the expression level of a protein encoded by the gene in respective cells, and the expression level capable of suppressing reduction of a recombinant protein is evaluated based on a difference therebetween.

The evaluation in the second step of the second embodiment is specifically preferably performed according to the following procedures (a) to (d):

(a) calculating a relative quantitative value by dividing the expression level of a protein encoded by the gene in a test cell measured in the first step by the expression level of a protein (standard protein) encoded by a standard gene in the test cell,

a relative quantitative value=(the expression level of a protein encoded by the gene in a test cell measured in the first step)/(the expression level of a standard protein)

(b) calculating a control value by dividing the expression level of a protein encoded by the gene in a control cell by the expression level of the standard protein in the control cell,

a control value=(the expression level of a protein encoded by the gene in a control cell)/(the expression level of the standard protein)

(c) calculating a relative expression level by dividing the relative quantitative value for the test cell calculated in (a) by the control value calculated in (b),

a relative expression level=[the relative quantitative value for the test cell calculated in (a)]/[the control value calculated in (b)]

(d) comparing the relative quantitative value calculated in (a) or the relative expression level calculated in (c) among the respective test cells, and selecting a cell in which each value is high or low.

The procedure (d) is more preferably performed according to the following procedure (d-1) or (d-2):

(d-1) comparing the relative quantitative value calculated in (a) among the respective test cells, and thereby selecting a cell in which the value is high (when a protein encoded by at least one gene selected from genes comprising any one of the base sequences represented by SEQ ID NOS: 1 to 9 or orthologous genes thereof is used) or low (when a protein encoded by at least one gene selected from genes comprising any one of the base sequences represented by SEQ ID NOS: 10 to 16 or orthologous genes thereof is used), or

(d-2) comparing the relative expression level calculated in (c) among the respective test cells, and thereby selecting a cell in which the value is high (when a protein encoded by at least one gene selected from genes comprising any one of the base sequences represented by SEQ ID NOS: 1 to 9 or orthologous genes thereof is used) or low (when a protein encoded by at least one gene selected from genes comprising any one of the base sequences represented by SEQ ID NOS: 10 to 16 or orthologous genes thereof is used).

As the cell in which the relative quantitative value is high, a cell in which the relative quantitative value is twice or more, for example, 5 times or more, 7 times or more, 10 times or more, 20 times or more, 25 times or more, 30 times or more, 50 times or more, or 100 times or more the relative quantitative value (also referred to as “control value”) of a cell (control cell) showing the smallest value among the test cells is exemplified.

In particular, as the cell in which the relative quantitative value is high, a cell in which the relative quantitative value is preferably 5 times or more, more preferably 10 times or more, further more preferably 20 times or more, still further more preferably 25 times or more, yet still further more preferably 30 times or more, even yet still further more preferably 50 times or more, and most preferably 100 times or more the control value is exemplified.

As the cell in which the relative quantitative value is low, a cell in which the relative quantitative value is ½ or less, preferably ⅕ or less, more preferably 1/10 or less, further more preferably 1/20 or less of the relative quantitative value (also referred to as “control value”) of a cell (control cell) showing the largest value among the test cells is exemplified.

As the cell in which the relative expression level is high, a cell in which the relative expression level is twice or more, for example, 5 times or more, 7 times or more, 10 times or more, 20 times or more, 25 times or more, 30 times or more, 50 times or more, or 100 times or more the relative expression level of a cell showing the smallest value among the test cells is exemplified.

In particular, as the cell in which the relative expression level is high, a cell in which the relative expression level is preferably 5 times or more, more preferably 10 times or more, further more preferably 20 times or more, still further more preferably 25 times or more, yet still further more preferably 30 times or more, even yet still further more preferably 50 times or more, and most preferably 100 times or more the relative expression level of a cell showing the smallest value among the test cells is exemplified.

As the cell in which the relative expression level is low, a cell in which the relative expression level is ½ or less, preferably ⅕ or less, more preferably 1/10 or less, further more preferably 1/20 or less of the relative expression level of a cell showing the largest value among the test cells is exemplified.

Further, the evaluation of the expression level capable of suppressing reduction of a recombinant protein in the second step of the second embodiment can also be performed according to the following procedures (e) to (g):

(e) calculating relative quantitative value obtained by dividing the expression level of a protein encoded by the gene in a test cell measured in the first step by the expression level of a protein (standard protein) encoded by a standard gene in the test cell,

a relative quantitative value=(the expression level of a protein encoded by the gene in a test cell measured in the first step)/(the expression level of a standard protein)

(f) comparing the relative quantitative value for the test cell calculated in (e) with a relative quantitative value calculated in the same manner for another cell, and

(g) comparing the relative quantitative value calculated in (e) among the respective test cells, and thereby selecting a cell in which the value is high (when genes represented by SEQ ID NOS: 1 to 9 are used) or low (when genes represented by SEQ ID NOS: 10 to 16 are used) is selected.

As the standard protein, any protein can be used without particular limitation as long as it is a protein generally expressed in a cell, and examples thereof include GAPDH (Glyceraldehyde-3-phosphate dehydrogenase), β-actin, β2-microglobulin, HPRT1 (Hypoxanthine phosphoribosyltransferase 1), globulin, ubiquitin and the like.

Further, the measurement of the expression level of the standard protein can be performed by the ELISA method or the like in the same manner as the expression level of a protein encoded by the above-mentioned gene. Preferably, GAPDH is used.

As the control cell, for example, a cell in which a recombinant protein is easily reduced is exemplified. As the control cell, a sample, which is derived from the same cell as the cell measured for the expression level of a protein encoded by the gene in the first step, but whose sampling time is different, may be used, or it may be a cell derived from a different cell. Further, as the control cell, one may be selected from the test cells, and in that case, any cell may be selected among the respective test cells.

Specifically, for example, in the case where the evaluation is performed using a protein encoded by at least one gene selected from genes comprising any one of the base sequences represented by SEQ ID NOS: 1 to 9 or orthologous genes thereof, when a cell showing the lowest relative quantitative value among the values of the respective test cells subjected to measurement is used as the control cell, the difference from that of the test cell to be selected becomes larger, and therefore, the analysis of the results is easier.

On the other hand, in the case where the evaluation is performed using a protein encoded by at least one gene selected from genes comprising any one of the base sequences represented by SEQ ID NOS: 10 to 16 or orthologous genes thereof, when a cell showing the highest relative quantitative value is used as the control cell, the difference from that of the test cell to be selected becomes larger, and therefore, the analysis of the results is easier.

That is, the control cell is preferably a cell showing the lowest relative quantitative value when a protein encoded by at least one gene selected from genes comprising any one of the base sequences represented by SEQ ID NOS: 1 to 9 or orthologous genes thereof is used, and is a cell showing the highest relative quantitative value when a protein encoded by at least one gene selected from genes comprising any one of the base sequences represented by SEQ ID NOS: 10 to 16 or orthologous genes thereof is used.

In the first embodiment or the second embodiment, a step of introducing at least one gene selected from genes comprising any one of the base sequences represented by SEQ ID NOS: 1 to 9 or orthologous genes thereof into a cell, thereby transforming the cell may be included before the first step.

In the present invention, the “introducing a gene” means that the target gene is present in the cell. For example, the target gene is present in the chromosome of the cell or the target gene is included in a vector present in the cell. As the target gene, a gene of a recombinant protein to be produced by a cell and/or at least one gene selected from genes comprising any one of the base sequences represented by SEQ ID NOS: 1 to 9 or orthologous genes thereof is exemplified.

The first embodiment or the second embodiment may comprise, before the first step, a step of introducing at least one gene selected from genes comprising any one of the base sequences represented by SEQ ID NOS: 1 to 9 or orthologous genes thereof into a cell, thereby transforming the cell, and a step of introducing a gene encoding a recombinant protein into a cell, thereby transforming the cell. Either of these steps may be performed first or these steps may be performed simultaneously. As a method for introducing the gene to transform the cell, the above-mentioned method is exemplified.

In the introduction of the target gene into a cell, for example, a known method such as an electroporation method, a calcium phosphate method, a liposome method, or a DEAE dextran method can be used. When a CHO cell is used as a host cell, for example, the target gene is introduced into an expression vector, and the resulting vector is transfected into the CHO cell by a lipofection method or the like, and the resulting cell is cultured, thereby the CHO cell transfected with the gene can be obtained.

As a method for introducing the target gene into a cell, an appropriate method can be used according to the cell, and for example, a method using an expression vector is exemplified. The expression vector is not particularly limited, and a plasmid vector is preferably used. Other than this, for example, a viral vector, a cosmid vector, a bacterial artificial chromosome (BAC), a yeast artificial chromosome (YAC), and other non-plasmid vectors may be used. As these vectors, commercially available vectors can also be used. Incidentally, when a cell derived from a mammal is used as the cell, as the expression vector, an expression vector comprising a promoter, a splicing region, a poly-A addition site, etc. is preferably used.

Further, in the first embodiment or the second embodiment, a step of knocking down or knocking out at least one gene selected from genes comprising any one of the base sequences represented by SEQ ID NOS: 10 to 16 or orthologous genes thereof in the cell, thereby downregulating the expression level of the gene may be included before the first step.

In the present invention, the “knocking down or knocking out” means that transcription or translation of the target gene is inhibited and reduced as compared with normal cells. By doing this, the function of the target gene is lost or attenuated.

In the knocking down or knocking out of the gene, any technique can be used as long as the function of the gene is suppressed or lost. For example, the expression level of the gene can be suppressed using a method for introducing an antisense RNA into a cell, an RNAi method using an siRNA, a microRNA, or the like, etc. Further, it is also possible to use a method for knocking out the gene using a genome-editing technique such as TALEN or CRISPR-Cas9.

The first embodiment or the second embodiment may comprise, before the first step, a step of knocking down or knocking out at least one gene selected from genes comprising any one of the base sequences represented by SEQ ID NOS: 10 to 16 or orthologous genes thereof in a cell, and a step of introducing a gene encoding a recombinant protein into a cell, thereby transforming the cell. Either of these steps may be performed first or these steps may be performed simultaneously. As a method for knocking down or knocking out the gene and a method for introducing the gene to transform the cell, the above-mentioned methods are exemplified.

There is a high possibility that the thus selected cell has an ability to suppress reduction of a target recombinant protein, and by using the cell as a host cell, a cell producing any recombinant protein can be obtained. The recombinant protein produced by the cell (production cell) obtained by the selection method of the present invention can be isolated without being degraded or denatured by reduction.

When a protein-producing cell is established, it is often the case that several tens to several thousands or more of cell populations are established, and an appropriate cell for producing a recombinant protein is selected therefrom. The production cell obtained based on the method of the present invention has a property of suppressing reduction of a recombinant protein. At least the probability that the production cell obtained based on the method of the present invention has a property of suppressing reduction of a recombinant protein is high. That is, according to the present invention, the probability of acquisition of a cell capable of suppressing reduction of a recombinant protein can be increased as compared with a conventional method.

The selection method of the present invention can be combined with a method for selecting a production cell based on another index such as productivity, high expression, or quality. Therefore, the present invention can provide a method for acquiring a cell highly producing or highly expressing a recombinant protein.

A third embodiment of the present invention is a cell capable of suppressing reduction of a recombinant protein, and acquisition thereof including introducing at least one gene selected from genes comprising any one of the base sequences represented by SEQ ID NOS: 1 to 9 or orthologous genes thereof into a cell, thereby transforming the cell.

A fourth embodiment of the present invention is a cell capable of suppressing reduction of a recombinant protein, in which at least one gene selected from genes comprising any one of the base sequences represented by SEQ ID NOS: 10 to 16 or orthologous genes thereof is knocked down or knocked out, and acquisition thereof.

By using a cell into which at least one gene selected from genes comprising any one of the base sequences represented by SEQ ID NOS: 1 to 9 or orthologous genes thereof is introduced, or a cell in which at least one gene selected from genes comprising any one of the base sequences represented by SEQ ID NOS: 10 to 16 or orthologous genes thereof is knocked down or knocked out as a host cell, reduction of a recombinant protein produced from the cell and degradation or denaturation accompanying this can be suppressed. Accordingly, a production cell in which decrease or loss of the activity of a recombinant protein is suppressed can be obtained.

By culturing the cell obtained according to the present invention in an ordinary manner, a target protein can be produced in the native form, that is, a form in which a disulfide bond is formed at a proper site. As the method for culturing the cell, a well-known method for producing a protein such as batch culture, fed-batch culture, or perfusion culture can be used. The obtained culture broth is subjected to purification after being subjected to a cell separation step. The cell separation step is performed by a method such as continuous centrifugation, batch centrifugation, filtration, or perfusion.

In order to isolate and purify the recombinant protein from the cell, known separation operations can be performed in combination. Specifically, for example, a treatment with a denaturing agent such as urea or a surfactant, a sonication treatment, enzymatic digestion, salt precipitation, a solvent fractional precipitation method, dialysis, centrifugation, ultrafiltration, gel filtration, SDS-PAGE, isoelectric focusing, ion exchange chromatography, hydrophobic chromatography, affinity chromatography, reverse-phase chromatography, and the like are exemplified, however, the method is not limited thereto.

### EXAMPLES

Next, the present invention will be described in more detail with reference to Examples, however, the invention is by no means limited thereto.

### Example 1

### Gene Expression Difference Analysis of Antibody-Producing Cell Using Microarray Analysis

A gene expression difference analysis using a DNA microarray was performed for the purpose of specifying a gene (mRNA) expressed correlatively with a difference in ease of reduction of a protein (antibody) to be produced.

Cells in which an antibody to be produced is not easily reduced [a CHO cell #1 producing an IgG4-type monoclonal antibody (hereinafter referred to as “antibody A”) (hereinafter abbreviated as “A #1 strain”), and a CHO cell #1 producing an IgG1/IgG3 chimeric monoclonal antibody (hereinafter referred to as “antibody B”) (hereinafter abbreviated as “B #1 strain”)] that is an antibody against an antigen different from the antibody A, and cells in which an antibody to be produced is easily reduced [a CHO cell #2 producing the antibody A (hereinafter abbreviated as “A #2 strain”), and a CHO cell #2 producing the antibody B (hereinafter abbreviated as “B #2 strain”)] were used for evaluation. The level of ease of reduction in each cell is shown in Table 1. Incidentally, evaluation of the ease of reduction was performed as follows.

That is, cells were inoculated in a 250-mL Erlenmeyer flask and cultured for 13 days in a CO₂ incubator. During the culture period, a feed medium was appropriately added, and also sampling was performed. A viable cell density and a viability were measured using a live/dead cell autoanalyzer (Vi-CELL XR), and the concentration of the antibody was measured using Protein A HPLC.

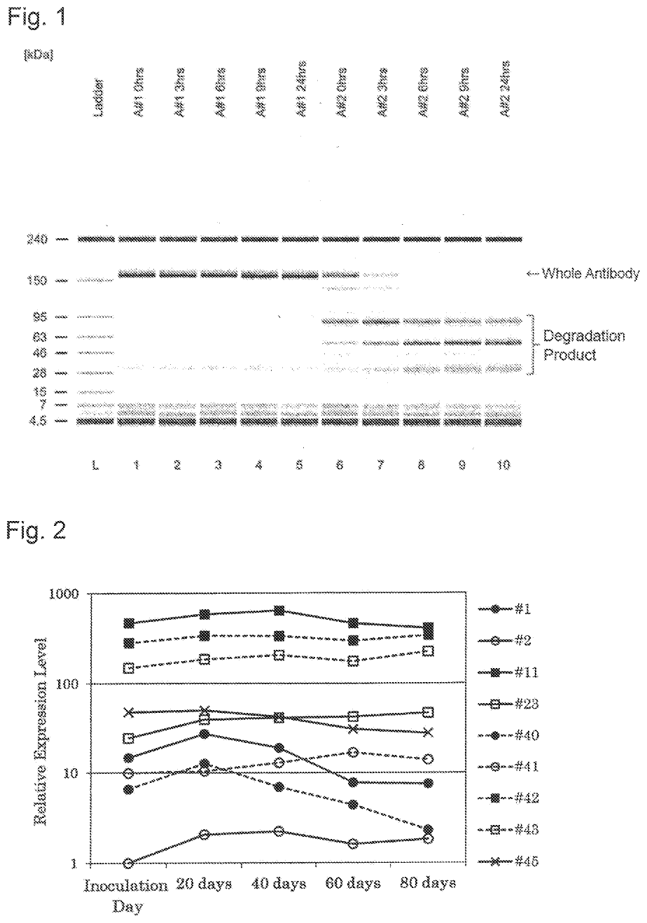

The cells on day 13 of culture were homogenized using an ultrasonic homogenizer (VP-300, manufactured by TAITEC Corporation), and a cell homogenate was incubated under an anaerobic condition. Sampling was performed from the solution over time, and capillary electrophoresis was performed using Agilent 2100 Bioanalyzer (manufactured by Agilent Co., Ltd.), and the degradation state of the produced protein was evaluated. The results for the A #1 strain and the A #2 strain are shown in FIG. 1.

As shown in FIG. 1, when changes in the structures of the antibodies produced by the A #1 strain and the A #2 strain were observed, it was confirmed that in the case of the A #1 strain, the band of the target product (Whole antibody, at around 150 kDa) did not change, however, in the case of the A #2 strain, the degradation products having a low molecular weight (28 to 95 kDa) were increased over time with the progress of the reduction reaction.

TABLE 1

| Production cell | Ease of reduction |
|---|---|
| A#1 | low |
| A#2 | high |
| B#1 | low |
| B#2 | high |

These cells were cultured for 14 days in a CO₂ incubator using a 250-mL Erlenmeyer flask. During the culture period, addition of a feed medium and sampling were appropriately performed. On day 3 and day 9 of culture, sampling was performed from each cell culture broth, and total RNA was extracted from about 1×10⁷ cells of each using ISOGEN (manufactured by Nippon Gene Co., Ltd.).

By using a 50 μg portion of the obtained total RNA, a DNase treatment was performed, and after a phenol-chloroform treatment and ethanol precipitation were performed, a microarray analysis (manufactured by Takara Bio, Inc.) was performed from a solution containing the total RNA in an amount of 1 μg or more for each using GENECHIP™ CHO Gene 2.0 ST Array (manufactured by Affymetrix, Inc.).

Data analysis was performed using GENESPRING™ GX (manufactured by Agilent Co., Ltd.), and a numerical value correction method was performed using the RMA method or the PLIER method (Yi Qu et al. BMC Bioinformatics, 211, 11, 2010).

By the analysis results, 29700 genes whose expression levels are different were confirmed. From the data created by each correction method, 16 genes whose expression level is different twice or more between the strain in which the antibody is not easily reduced and the strain in which the antibody is easily reduced, were specified. The list of the 16 genes are shown in Table 2.

TABLE 2

| | | | Ratio of Expression Level |
|---|---|---|---|
| | | | (cell strain with low |
| | | | susceptibility to |
| Transcript Cluster | NCBI Reference | | reduction/cell with high |
| Sequence ID | Sequence ID | Description of Gene | susceptibility to reduction) |
| 18083239 | XM_003498539.3:95-718 | placenta-expressed transcript 1 | High expression, |
| | | protein | twice or more |
| 17952793 | | uncharacterized | |
| 17952795 | | uncharacterized | |
| 17952194 | XM_003504576.3:16-1887 | matrilin 4 (Matn4) | |
| 17957314 | | uncharacterized | |
| 17958207 | | uncharacterized | |
| 17968846 | XM_007653651.2:513-3224 | G-protein-coupled receptor 133 | |
| 18020489 | XM_003506418.3:373-6432 | tenascin C | |
| 18024962 | | uncharacterized | |
| 17955459 | XM_003515418.2:264-4664 | collagen alpha-1(III) chain | Low expression, |
| 17967811 | XM_003513514.3:198-863 | glutathione S-transferase alpha-3 | half or less |
| 18038891 | XM_003503929.3:51-1874 | calcium/calmodulin-dependent | |
| | | 3′,5′-cyclic nucleotide | |
| | | phosphodiesterase 1C | |
| 18042016 | XM_007644176.2:86-1984 | anthrax toxin receptor 1 | |
| and | | | |
| 18113632 | | | |
| 18042016 | XM_003503574.1 | gastrokine-1 | |
| and | | | |
| 18113632 | | | |
| 18056819 | XM_003501810.3 | tumor necrosis factor ligand | |
| | | superfamily member 9 | |
| 18066454 | XM_003500554.3:188-3268 | collagen, type VI, alpha 2 | |

It is considered that by using the expression level of the specified gene shown in Table 2 or the production amount of the protein encoded thereby as an index, a cell in which a reducing activity for a target protein such as an antibody is different can be specified.

### Example 2

### Determination of Ease of Reduction of Production Cell by Expression Difference Analysis of Placenta-Expressed Transcript 1 Protein (Hereinafter Referred to as Plet1) Gene Using RT-qPCR Method

The following analysis was performed using Plet1 gene that was revealed to be highly expressed in a production strain in which a protein to be produced is not easily reduced among the genes found in Example 1 as an example.

With respect to the total RNA obtained from four types of cells (Table 1) prepared in Example 1 and used in the microarray analysis, the expression level of a gene was analyzed by the following method using the RT-qPCR method. First, based on the information of the base sequence of Plet1 gene of a Chinese hamster, a probe and primers to be used in the RT-qPCR method were designed (Table 3, SEQ ID NOS: 3 to 5).

A reaction solution was prepared as 20 μL of a reaction system containing 5 μL of TAQMAN™ Fast Virus 1-Step Master Mix (manufactured by Thermo Fisher Scientific, Inc.) and using the probe at 200 nM (final concentration), the primers at 500 nM (final concentration), and 10 ng of the total RNA per reaction. The reaction was performed using a real-time PCR device (7900HT Fast Real Time PCR System, manufactured by Applied Biosystems, Inc.) according to a schedule in which after a pre-reaction at 50° C. for 5 minutes→95° C. for 20 seconds, a cycle of “95° C. for 3 seconds→60° C. for 30 seconds” is repeated 40 times.

The gene expression level of Plet1 was compared among cells using a relative expression level. Specifically, first, a relative quantitative value corrected by dividing the gene expression level of Plet1 by the gene expression level of GAPDH was calculated. Subsequently, a cell showing the lowest relative quantitative value was used as a control cell, and a relative expression level was calculated by dividing the relative quantitative value of each of the other cells by the relative quantitative value (control value) of the control cell. Incidentally, GAPDH used here is one example of a housekeeping gene generally expressed in a cell. The evaluation results are shown in Table 4.

TABLE 3

| Name | Base Sequence |
|---|---|
| Probe (obtained by attaching | 5′-FAM-caatctccaccag |
| FAM to the 5′ end of SEQ ID | cgac-MGB-3′ |
| NO: 28 and attaching MGB to | |
| to the 3′ end thereof) | |
| Forward Primer | 5′-gacccctgcatggtcttt |
| (SEQ ID NO: 29) | ga-3′ |
| Reverse Primer | 5′-accgtaatgctgactccc |
| (SEQ ID NO: 30) | aagt-3′ |

TABLE 4

| | |
|---|---|
| Sampling day | Relative expression level |

| | | |
|---|---|---|
| Day 3 | A#1 strain | 97 |
| | A#2 strain | 1 |
| | B#1 strain | 208 |
| | B#2 strain | 1 |
| Day 9 | A#1 strain | 84 |
| | A#2 strain | 1 |
| | B#1 strain | 52 |
| | B#2 strain | 1 |

As shown in Table 4, it was confirmed that in the cells in which the antibody is not easily reduced (A #1 strain and B #1 strain), the relative expression level of Plet1 gene is 52 to 208 times higher than in the cells in which the antibody is easily reduced (A #2 strain and B #2 strain).

From this result, it is considered that by quantitatively determining the expression level of mRNA of Plet1 gene using a quantitative gene analysis method such as the RT-qPCR method, a production cell in which a target protein is not easily reduced can be easily specified.

### Example 3

### Selection of Production Cell in which Protein is Not Easily Reduced by Expression Difference Analysis of Plet1 Gene

Chinese hamster ovary-derived cells (CHO cells) were acclimated in a serum-free medium, whereby host cells X were obtained. By using the method described in U.S. Pat. No. 6,946,292, CHO cells in which fucosyltransferase (FUT8) was deleted were obtained and used as host cells Y.

Subsequently, the host cells Y were suspended in PBS, and gene transfer was performed using an expression vector integrated with a gene of an IgG1-type monoclonal antibody (hereinafter referred to as “antibody C”) that is an antibody against an antigen different from the antibody A or the antibody B.

After gene transfer by electroporation, the cells in a cuvette were suspended in a medium and inoculated into a 96-well plate and cultured in a CO₂ incubator for a few days. Subsequently, after a few days from the gene transfer, the medium was exchanged with a medium containing cycloheximide and the cells were cultured for 4 weeks while performing medium exchange once a week. During that period, the size of the plate was appropriately changed, and the cells were grown to a scale of a 125-mL volume Erlenmeyer flask in the end. The culture supernatant was sampled, and cells which produced the antibody in a large amount were selected and subjected to single-cell cloning.

Subsequently, the obtained cells were inoculated into a 384-well plate using a flow cytometer (FACS Aria II, manufactured by Becton, Dickinson and Company) or a limiting dilution method. Culture was continued, and cells were obtained from a well in which a single colony was formed, and the cells were subjected to extended culture to a scale of a 125-mL volume Erlenmeyer flask.

From these cells, cells were selected using the productivity of the antibody as an index, and 46 types of cells were obtained, whereby 46 strains of antibody C-producing cells were prepared. The 46 strains of antibody C-producing cells were named antibody C-producing strain #1 to #46, respectively. The cells of each strain were dispensed in a 1-mL vial and cryopreserved, respectively.

Subsequently, the respective cells were cultured for 14 days in a CO₂ incubator using a 250-mL volume Erlenmeyer flask. During the culture period, a feed medium was appropriately added, and also sampling was performed. On day 3 of culture, sampling was performed from each cell culture broth (1 mL each), and a cell pellet (about 3×10⁶ cells) recovered by centrifugation was dissolved by adding 500 μL of ISOGEN, whereby total RNA was obtained.

Subsequently, by using this, the gene expression level of Plet1 was measured by the RT-qPCR method in the same manner as in Example 2. At that time, GAPDH was selected as one example of a standard gene, and a relative quantitative value of each antibody C-producing strain with respect to the expression level of GAPDH was calculated. Further, as one example, the antibody C-producing strain #5 was used as a control cell. By assuming the relative quantitative value of Plet1 of the antibody C-producing strain #5 to be 1.0, the relative expression level of Plet1 of each cell was calculated. Further, on day 14, the ease of reduction of the antibody to be produced was evaluated for each cell in the same manner as in Example 1. The results are shown in Table 5.

TABLE 5

| Antibody C-producing | Relative expression | Whether antibody |
|---|---|---|
| strain | level | was reduced or not |
| #1 | 14.9 | reduced |
| #2 | 2.0 | reduced |
| #3 | 2.6 | reduced |
| #4 | 1.4 | reduced |
| #5 | 1.0 | reduced |
| #6 | 2.5 | reduced |
| #7 | 2.6 | reduced |
| #8 | 1.6 | reduced |
| #9 | 1.5 | reduced |
| #10 | 2.3 | reduced |
| #11 | 675.6 | not reduced |
| #12 | 955.4 | not reduced |
| #13 | 3.2 | reduced |
| #14 | 2.8 | reduced |
| #15 | 5.3 | reduced |
| #16 | 1.9 | reduced |
| #17 | 2.8 | reduced |
| #18 | 10.6 | reduced |
| #19 | 3.0 | reduced |
| #20 | 8.0 | reduced |
| #21 | 36.8 | not reduced |
| #22 | 724.1 | reduced |
| #23 | 36.8 | not reduced |
| #24 | 18.4 | reduced |
| #25 | 9.8 | reduced |
| #26 | 1.6 | reduced |
| #27 | 1.7 | reduced |
| #28 | 1.2 | reduced |
| #29 | 1.6 | reduced |
| #30 | 2.0 | reduced |
| #31 | 2.6 | reduced |
| #32 | 4.0 | reduced |
| #33 | 8.6 | reduced |
| #34 | 1.7 | reduced |
| #35 | 2.0 | reduced |
| #36 | 2.6 | reduced |
| #37 | 0.0 | reduced |
| #38 | 4.3 | not reduced |
| #39 | 1.0 | not reduced |
| #40 | 4.9 | reduced |
| #41 | 7.5 | reduced |
| #42 | 388.0 | not reduced |
| #43 | 222.9 | not reduced |
| #44 | 21.1 | not reduced |
| #45 | 48.5 | not reduced |
| #46 | 362.0 | not reduced |

There were 11 strains (24%) of cells in which the antibody was not reduced (#11, #12, #21, #23, #38, #39, #42, #43, #44, #45, and #46) among the 46 cells of the antibody C-producing strains. On the other hand, there existed 18 production strains in which the relative expression level of Plet1 is 5 times or more higher than that of the antibody C-producing strain #5 (#1, #11, #12, #15, #18, #20 to #25, #33, and #41 to #46), and among these, there existed 10 production strains (56%) in which the antibody was not reduced (#11, #12, #21, #23, and #41 to #46). There existed 13 strains of production cells in which the relative expression level of Plet1 is 10 times or more higher than that of the antibody C-producing strain #5 (#1, #11, #12, #18, #21 to #24, and #42 to #46), and among these, there existed 9 strains (69%) of cells in which the antibody was not reduced.

Further, there existed 11 strains of production cells in which the relative expression level of Plet1 is 15 times or more higher than that of the antibody C-producing strain #5, and among these, there existed 9 strains (82%) of cells in which the antibody was not reduced (#11, #12, #21, #23, and #42 to #46). In addition, there existed 6 strains of production cells in which the relative expression level of Plet1 is 100 times or more higher than that of the antibody C-producing strain #5 (#11, #12, #22, #42, #43, and #46), and among these, there existed 5 strains (83%) of cells in which the antibody was not reduced (#11, #12, #42, #43, and #46).

From this result, it was confirmed that by selecting a cell in which the expression level of Plet1 gene is high using the RT-qPCR method, a cell in which a target protein is not reduced can be obtained with high probability. Incidentally, it was also shown that by arbitrarily increasing the relative expression level of Plet1 gene, for example, from 5 times to 100 times as compared with the control value, the probability that a target protein is not reduced can be increased.

### Example 4

### Confirmation of Versatility of Selection of Production Cell in which Target Protein is not Easily Reduced by Expression Difference Analysis of Plet1 Gene

For the purpose of confirming the versatility of the method for specifying a production strain in which a target protein is not easily reduced by an expression difference analysis of Plet1 gene, an examination was performed using antibody D-producing cells.

According to the method shown in Example 3, a gene of an IgG4-type monoclonal antibody (hereinafter referred to as “antibody D”) that is an antibody against an antigen different from the antibody A, the antibody B, or the antibody C was introduced into CHO cells, followed by single-cell cloning, whereby 6 strains of antibody D-producing cells were prepared and named antibody D-producing strain #1 to #6, respectively.

The respective cells were cultured for 14 days in a CO₂ incubator using a 250-mL volume Erlenmeyer flask. During the culture period, a feed medium was appropriately added, and also sampling was performed. On day 14 of culture, the ease of reduction of the antibody to be produced was evaluated in the completely same manner as in Example 1. Subsequently, a cryopreserved stock prepared during subculture of each cell was thawed, and a cell pellet (about 3×10⁶ cells) recovered by centrifugation was dissolved by adding 500 μL of ISOGEN, whereby total RNA was obtained.

By using this, the gene expression level of Plet1 was measured by the RT-qPCR method in the same manner as in Example 2. GAPDH was selected as one example of a standard gene, and a relative quantitative value corrected by dividing the gene expression level of Plet1 by the gene expression level of GAPDH was calculated. Further, one cell (the antibody D-producing strain #5) was selected as a control cell, and by using the relative quantitative value of the strain (the antibody D-producing strain #5) as a control value, the relative expression level of Plet1 of each of the other cells was calculated. The results of the gene expression level of Plet1 in the antibody D-producing strains and evaluation of reduction of the antibody were compared. The results are shown in Table 6.

TABLE 6

| Antibody D-producing | Relative expression | Whether antibody |
|---|---|---|
| strain | level | was reduced or not |
| #1 | 24.9 | not reduced |
| #2 | 42.5 | not reduced |
| #3 | 1.2 | reduced |
| #4 | 70.8 | reduced |
| #5 | 1.0 | reduced |
| #6 | 7.7 | not reduced |

As shown in Table 6, there were 3 strains (50%) of cells in which the antibody was not reduced (#1, #2, and #6) among the 6 antibody D-producing strains. There existed 4 strains of production cells in which the relative expression level of Plet1 gene is 5 times or more higher than that of the antibody D-producing strain #5 (#1, #2, #4, and #6), and among these, there existed 3 strains (75%) of cells in which the antibody was not reduced (#1, #2, and #6). As described above, it was confirmed that by analyzing the gene expression level of Plet1, a production strain in which reduction does not easily occur can be obtained regardless of the type of the antibody or the production strain.

### Example 5

### Expression Analysis of Plet1 Gene Using Cell During Subculture

Among the 46 cells of the antibody C-producing strains for which evaluation was performed in Example 3, 9 cells in which the gene expression level of Plet1 is different were subcultured for 80 days, and the relative expression level of Plet1 gene was analyzed. The results are shown in FIG. 2.

As shown in FIG. 2, in the 80 days of subculture period, the expression level of Plet1 gene was stable. As described above, even if the number of generation of the cells was changed, the expression level of Plet1 gene was constant, and therefore, it was confirmed that a cell in which the ease of reduction is different can be selected using Plet1 as an index without depending on the state of the cell such as the number of generations.

### Example 6

### Acquisition of Host Cells (CHO Cells X′ and Y′) in which Recombinant Protein is not Easily Reduced

By using the two types of host cells (X and Y) obtained in Example 3, single-cell cloning was performed by the following method, whereby subcloning strains were produced.

Each cell (X or Y) was subjected to extended culture to a scale of a 125-mL Erlenmeyer flask. Subsequently, by using Vi-CELL, a viable cell density was measured, and a cell solution was prepared so as to contain about 16 cells per 100 well. The cell solution was inoculated into a 384-well plate in an amount of 50 μL each, and the cells were cultured. The cells in a well in which a single colony was formed were cultured and grown to a scale of a 125-mL volume Erlenmeyer flask, and thereafter, the cells were dispensed in an amount of 1 mL each and cryopreserved.

By using the cells obtained during culture in the above-mentioned process, an expression analysis of Plet1 gene was performed by the RT-qPCR method in the same manner as in Example 2. With respect to 40 strains of each of the subcloning strains of the host cells X and Y, a strain showing the lowest gene expression level of Plet1 was used as a control cell, and the relative expression level was calculated in the same manner as the above-mentioned Examples.

16 strains in which the gene expression level of Plet1 is high were obtained for each (X′ strain and Y′ strain). Further, as comparison subjects, two strains for each (Xc strains and Yc strains) in which the relative expression level of Plet1 is low were obtained as control cells. The results are shown in Table 7.

TABLE 7

| | Relative | | Relative |
|---|---|---|---|
| | expression | | expression |
| Host cell | level | Host cell | level |
| X′#1 | 22 | Y′#1 | 2 |
| X′#2 | 52 | Y′#2 | 18 |
| X′#3 | 21 | Y′#3 | 5 |
| X′#4 | 21 | Y′#4 | 9 |
| X′#5 | 111 | Y′#5 | 20 |
| X′#6 | 110 | Y′#6 | 31 |
| X′#7 | 84 | Y′#7 | 12 |
| X′#8 | 23 | Y′#8 | 20 |
| X′#9 | 36 | Y′#9 | 6 |
| X′#10 | 17 | Y′#10 | 65 |
| X′#11 | 28 | Y′#11 | 13 |
| X′#12 | 45 | Y′#12 | 17 |
| X′#13 | 92 | Y′#13 | 24 |
| X′#14 | 56 | Y′#14 | 5 |
| X′#15 | 57 | Y′#15 | 13 |
| X′#16 | 22 | Y′#16 | 15 |
| Xc#1 | 1 | Yc#1 | 1 |
| (standard strain) | | (standard strain) | |
| Xc#2 | 4 | Yc#2 | 1 |

As shown in Table 7, in the case of the X′ strain, there existed 9 strains in which the relative expression level of Plet1 gene is 30 times or more higher than that of the Xc #1 strain (X′ #2, X′ #5, X′ #6, X′ #7, X′ #9, and X′ #12 to X′ #15), and there existed 7 strains in which the relative expression level of Plet1 gene is 50 times or more higher than that of the Xc #1 strain (X′ #2, X′ #5, X′ #6, X′ #7, and X′ #13 to X′ #15). In the case of the Y′ strain, there existed 11 strains in which the relative expression level of Plet1 gene is 10 times or more higher than those of the Yc #1 strain and Yc #2 strain (Y′ #2, Y′ #5 to Y′ #8, X′ #10 to Y′ #13, Y′ #15, and Y′ #16), and there existed 5 strains in which the relative expression level of Plet1 gene is 20 times or more higher than those of the Yc #1 strain and Yc #2 strain (Y′ #5, Y′ #6, Y′ #8, X′ #10, and Y′ #13). The selected strains were further scaled up to a 125-mL volume Erlenmeyer flask, and thereafter dispensed and cryopreserved.

It is considered that if the relative expression level of Plet1 gene of a host cell itself can be increased as in the case of the host cells obtained in the Example, production cells in which a recombinant protein is not easily reduced can be easily obtained.

### Example 7

### Confirmation that Production Strain Using Host Cell Highly Expressing Plet1 can Produce Antibody that is not Reduced

It was thought that a cell producing a protein that is not easily reduced can be prepared by using a host cell in which the gene expression level of Plet1 is increased, and therefore, the following test was performed.

With respect to the host cells in which the expression level of Plet1 is high (X′ #5, X′ #6, X′ #13, and Y′ #10) obtained in Example 6, antibody-producing strains (BX′ #1, BX′ #2, BX′ #3, and BY′ #1) into which the antibody B was introduced were obtained according to the method shown in Example 3.

Further, in order to confirm the difficulty in reduction of these antibody-producing strains, also with respect to host cells (Xc #2 and Yc #1) in which the expression level of Plet1 is low as controls, the same procedure was performed, whereby antibody B-producing strains BXc #1 and BYc #1 were obtained.

The above-prepared strains were cultured for 13 days in a CO₂ incubator using a 125-mL volume Erlenmeyer flask. During the culture period, a feed medium was appropriately added. The extraction of RNA was performed using mRNA catcher (Thermo Fisher Scientific, Inc.) for 50 μL of the culture broth on day 6 for each culture broth.

With respect to the obtained mRNA, the gene expression level of Plet1 was measured using the RT-qPCR method in the same manner as in Example 2. With respect to three strains of each of the BX′ strain and the BY′ strain, the relative expression level (Fold change) was calculated using the BXc #1 and BYc #1 showing the lowest Plet1 gene expression level as control cells. Further, on day 13 of culture, the susceptibility to reduction of the antibody was evaluated in the same manner as in Example 1.

The results are shown in Table 8 and Table 9.

TABLE 8

| | Antibody- | Relative | |
|---|---|---|---|
| | producing | expression | Whether antibody |
| Host | strain | level | was reduced or not |
| X′#5 | BX′#1 | 7 | not reduced |
| (High expression of Plet1) | | | |
| X′#6 | BX′#2 | 8 | not reduced |
| (High expression of Plet1) | | | |
| X′#13 | BX′#3 | 7 | not reduced |
| (High expression of Plet1) | | | |
| Xc#2 | BXc#1 | 1 | reduced |
| (Low expression of Plet1) | | | |

TABLE 9

| | Antibody- | Relative | |
|---|---|---|---|
| | producing | expression | Whether antibody |
| Host | strain | level | was reduced or not |
| Y′#10 | BY′#1 | 25 | not reduced |
| (High expression of Plet1) | | | |
| Yc#1 | BYc#1 | 1 | reduced |
| (Low expression of Plet1) | | | |

As shown in Table 8, in the three strains (BX′ #1, BX′ #2, and BX′ #3) prepared from the host cell X′ strain in which the relative expression level of Plet1 is high, reduction was not observed in all the antibody-producing strains. On the other hand, in one strain (BXc #1) prepared from the Xc strain in which the relative expression level of Plet1 is low, reduction of the antibody was confirmed.

Similarly, as shown in Table 9, also in the BY′ #1 strain prepared from the host cell BY′ #10 strain in which the relative expression level of Plet1 is high, reduction of the antibody was not observed. On the other hand, in one strain (BYc #1) prepared from the Yc strain in which the relative expression level of Plet1 is low, reduction of the antibody was confirmed.

From the above results, it was indicated that by using a host cell in which the relative expression level of Plet1 gene is increased, a cell producing an antibody that is less susceptible to reduction can be obtained with high probability.

While the present invention has been described in detail and with reference to specific embodiments thereof, it will be apparent to one skill in the art that various changes and modifications can be made therein without departing from the spirit and scope thereof. This application is based on Japanese Patent Application (Japanese Patent Application No. 2016-256279) filed on Dec. 28, 2016, the entire contents of which are incorporated hereinto by reference.

1. A method for selecting a cell and producing a recombinant protein employing the selected cell, comprising the following steps:
(a) measuring expression level of Plet1 gene and a standard gene in test cells and expression level of Plet1 gene and a standard gene in a control cell, the Plet1 gene comprising the nucleotide sequence of SEQ ID NO: 1;
(b) calculating a relative quantitative value by dividing the expression level of the Plet1 gene in the test cells measured in the step (a) by the expression level of the standard gene in the test cells measured in the step (a):
relative quantitative value=[the expression level of the Plet1 gene in the test cells measured in the step (a)]/[the expression level of the standard gene in the test cells measured in the step (a)];
(c) calculating a control value by dividing the expression level of the Plet1 gene in the control cell measured in the step (a) by the expression level of the standard gene in the control cell measured in the step (a):
control value=[the expression level of the Plet1 gene in the control cell measured in the step (a)]/[the expression level of the standard gene in the control cell measured in the step (a)];
(d) calculating a relative expression level by dividing the relative quantitative value for the test cells calculated in the step (b) by the control value calculated in the step (c):
relative expression level=[the relative quantitative value for the test cells calculated in the step (b)]/[the control value calculated in the step (c)]; and
(e) selecting the test cell in which the relative quantitative value calculated in the step (b), or relative expression level calculated in the step (d), is high relative to that of the other test cells; and
(f) using the selected test cell to produce a recombinant protein;
wherein the test cells and the control cell are a Chinese hamster ovary (CHO) cell.

2. The method according to claim 1, further comprising the step of introducing a gene encoding a recombinant protein to the test cells and the control cell, before the step (a).

3. The method according to claim 1, wherein the recombinant protein is an antibody.

4. The method according to claim 1, wherein the test cells and the control cell comprise a gene encoding a recombinant protein.

5. The method according to claim 4, wherein the recombinant protein is an antibody.

6. The method according to claim 1, wherein in the step (e), the cell in which the relative quantitative value or the relative expression level is high is a cell in which the relative quantitative value is twice or more the control value or the relative expression level is two or more.

7. The method according to claim 1, wherein the step (f) comprises culturing the selected test cell under a condition for expressing the recombinant protein.

8. The method according to claim 7, wherein the recombinant protein is an antibody.
